SEQUENCE LISTING (6 sequences)

SEQ ID NO: 1 - protein, 496 aa, Homo sapiens
MRVLSGTSLMLCSLLLLLQALCSPGLAPQSRGHLCRTRPTDLVFVVDSSRSVRPVEFEKVKVFLSQVIESLDVGPNATRVGMVNYASTVKQEFSLRAHVSKAALLQAVRRIQPLSTGTMTGLAIQFAITKAFGDAEGGRSRSPDISKVVIVVTDGRPQDSVQDVSARARASGVELFAIGVGSVDKATLRQIASEPQDEHVDYVESYSVIEKLSRKFQEAFCVVSDLCATGDHDCEQVCISSPGSYTCACHEGFTLNSDGKTCNVCSGGGGSSATDLVFLIDGSKSVRPENFELVKKFISQIVDTLDVSDKLAQVGLVQYSSSVRQEFPLGRFHTKKDIKAAVRNMSYMEKGTMTGAALKYLIDNSFTVSSGARPGAQKVGIVFTDGRSQDYINDAAKKAKDLGFKMFAVGVGNAVEDELREIASEPVAEHYFYTADFKTINQIGKKLQKKICVEEDPCACESLVKFQAKVEGLLQALTRKLEAVSKRLAILENTVV

SEQ ID NO: 2 - protein, 493 aa, Gallus gallus
MDGIFCALPLSLLLLLQSCGVWGAPPQPRGTLCRTKPTDLVFIIDSSRSVRPQEFEKVKVFLSRVIEGLDVGPNSTRVGVINYASAVKNEFSLKTHQTKAELLQAVQRIEPLSTGTMTGLAIQFAISRAFSDTEGARLRSPNINKVAIVVTDGRPQDGVQDVSARARQAGIEIFAIGVGRVDMHTLRQIASEPLDDHVDYVESYSVIEKLTHKFQEAFCVVSDLCATGDHDCEQICISTPGSYKCACKEGFTLNNDGKTCSACSGGSGSALDLVFLIDGSKSVRPENFELVKKFINQIVESLEVSEKQAQVGLVQYSSSVRQEFPLGQFKNKKDIKAAVKKMAYMEKGTMTGQALKYLVDSSFSIANGARPGVPKVGIVFTDGRSQDYITDAAKKAKDLGFRMFAVGVGNAVEDELREIASEPVAEHYFYTADFRTISNIGKKLQMKICVEEDPCECKSIVKFQTKVEELINTLQQKLEAVAKRIEALENKII

SEQ ID NO: 3 - protein, 956 aa, Homo sapiens
MEKMLAGCFLLILGQIVLLPAEARERSRGRSISRGRHARTHPQTALLESSCENKRADLVFIIDSSRSVNTHDYAKVKEFIVDILQFLDIGPDVTRVGLLQYGSTVKNEFSLKTFKRKSEVERAVKRMRHLSTGTMTGLAIQYALNIAFSEAEGARPLRENVPRVIMIVTDGRPQDSVAEVAAKARDTGILIFAIGVGQVDFNTLKSIGSEPHEDHVFLVANFSQIETLTSVFQKKLCTAHMCSTLEHNCAHFCINIPGSYVCRCKQGYILNSDQTTCRIQDLCAMEDHNCEQLCVNVPGSFVCQCYSGYALAEDGKRCVAVDYCASENHGCEHECVNADGSYLCQCHEGFALNPDKKTCTKIDYCASSNHGCQHECVNTDDSYSCHCLKGFTLNPDKKTCRRINYCALNKPGCEHECVNMEESYYCRCHRGYTLDPNGKTCSRVDHCAQQDHGCEQLCLNTEDSFVCQCSEGFLINEDLKTCSRVDYCLLSDHGCEYSCVNMDRSFACQCPEGHVLRSDGKTCAKLDSCALGDHGCEHSCVSSEDSFVCQCFEGYILREDGKTCRRKDVCQAIDHGCEHICVNSDDSYTCECLEGFRLAEDGKRCRRKDVCKSTHHGCEHICVNNGNSYICKCSEGFVLAEDGRRCKKCTEGPIDLVFVIDGSKSLGEENFEVVKQFVTGIIDSLTISPKAARVGLLQYSTQVHTEFTLRNFNSAKDMKKAVAHMKYMGKGSMTGLALKHMFERSFTQGEGARPLSTRVPRAAIVFTDGRAQDDVSEWASKAKANGITMYAVGVGKAIEEELQEIASEPTNKHLFYAEDFSTMDEISEKLKKGICEALEDSDGRQDSPAGELPKTVQQPTESEPVTINIQDLLSCSNFAVQHRYLFEEDNLLRSTQKLSHSTKPSGSPLEEKHDQCKCENLIMFQNLANEEVRKLTQRLEEMTQRMEALENRLRYR

SEQ ID NO: 4 - protein, 486 aa, Homo sapiens
MPRPAPARRLPGLLLLLWPLLLLPSAAPDPVARPGFRRLETRGPGGSPGRRPSPAAPDGAPASGTSEPGRARGAGVCKSRPLDLVFIIDSSRSVRPLEFTKVKTFVSRIIDTLDIGPADTRVAVVNYASTVKIEFQLQAYTDKQSLKQAVGRITPLSTGTMSGLAIQTAMDEAFTVEAGAREPSSNIPKVAIIVTDGRPQDQVNEVAARAQASGIELYAVGVDRADMASLKMMASEPLEEHVFYVETYGVIEKLSSRFQETFCALDPCVLGTHQCQHVCISDGEGKHHCECSQGYTLNADKKTCSALDRCALNTHGCEHICVNDRSGSYHCECYEGYTLNEDRKTCSAQDKCALGTHGCQHICVNDRTGSHHCECYEGYTLNADKKTCSVRDKCALGSHGCQHICVSDGAASYHCDCYPGYTLNEDKKTCSATEEARRLVSTEDACGCEATLAFQDKVSSYLQRLNTKLDDILEKLKINEYGQIHR

SEQ ID NO: 5 - protein, 581 aa, Homo sapiens [sequence omitted]

SEQ ID NO: 6 - protein, 40 aa, Artificial Sequence
  Description of Artificial Sequence Synthetic linker peptide
GGGGSGGGGSGGGGSGGGGSGGGGSGGGGSGGGGSGGGGS

(19) United States
(12) Patent Application Publication
(10) Pub. No.: US 2009/0203590 A1
(43) Pub. Date: Aug. 13, 2009

(54) METHOD FOR THE INHIBITION OF ANGIOGENESIS

(75) Inventors: Marsha A. Moses, Brookline, MA (US); Matthew J. Foradori, Edinboro, PA (US); Robert S. Langer, Newton, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, BOSTON, MA (US)

(21) Appl. No.: 11/915,417

(22) PCT Filed: May 30, 2006
(86) PCT No.: PCT/US2006/020932

§ 371 Date: Oct. 20, 2008

Related U.S. Application Data
(60) Provisional application No. 60/685,636, filed on May 27, 2005.

(51) Int. Cl. A61K 38/17; A61P 35/00

(52) U.S. Cl. 514/12

(57) ABSTRACT

The present invention is based on the discovery that Matrilin-1 has antiangiogenic and anticancer properties. The invention is directed to a method of treating a disease that responds to an inhibition of angiogenesis. Additionally, the invention can be applied to those at risk for developing a disease that responds to an inhibition of angiogenesis. The methods comprise administering to a mammal an effective angiogenesis-inhibiting amount of an Matrilin-1 consisting of Matrilin-1, Matrilin-1 fragment, analog, or derivative that is administered in a composition substantially free of other cartilage proteins.

### CROSS-REFERENCE TO RELATED APPLICATIONS

This application claims the benefit under 35 U.S.C. §119(e) of U.S. Provisional Patent Application No. 60/685,636 filed May 27, 2005.

### GOVERNMENT SUPPORT

This invention was made with Government Support under Contract No. AT00650 awarded by the National Institutes of Health. The Government has certain rights in the invention.

### FIELD OF INVENTION

The present invention relates to a method for treatment of cancer or diseases/disorders involving angiogenesis.

### BACKGROUND OF THE INVENTION

Angiogenesis is a process of tissue vascularization that involves the growth of new blood vessels into a tissue, and is also referred to as neo-vascularization. Blood vessels are the means by which oxygen and nutrients are supplied to living tissues and waste products are removed from living tissue. When appropriate, angiogenesis is a critical biological process. For example, angiogenesis is essential in reproduction, development and wound repair. Conversely, inappropriate angiogenesis can have severe negative consequences. For example, it is only after solid tumors are vascularized as a result of angiogenesis that the tumors have a sufficient supply of oxygen and nutrients that permit it to grow rapidly and metastasize.

In a recent review by Folkman, it was estimated that more than one-third of all women between the ages of 40 and 50 have in-situ tumors in their breasts. Such tumors lie dormant in the body and rarely, if ever, are diagnosed as breast cancer. It is believed that a similar phenomenon exists in men in regards to prostate cancer. In light of such data, cancer might be defined as having two distinct phases: (1) Acquisition of mutations which transform normal cells into cancerous cells, and the formation of in-situ tumors; and (2) A switch to an angiogenic phenotype, whereby the in-situ tumor is supplied with new blood vessels, supporting rapid tumor growth and metastasis (Nature, Vol. 427, Feb. 26, 2004, p. 787). Therapeutic compounds that are able to prevent the switch to an angiogenic phenotype (i.e. from an in-situ tumor to a rapidly growing tumor), are needed to prevent the onset of tumor growth. Angiogenesis inhibitors have shown promise in animal studies and clinical trials are currently underway (Kerbel et al. Nature Reviews, Vol. 2, pp. 727-739). However, new compounds that inhibit angiogenesis are needed.

### SUMMARY

The present invention is based, in part, on the discovery that a protein found in extract of shark cartilage, Matrilin-1, has antiangiogenic properties. The invention is directed to a method for treating a disease that responds to an inhibition of angiogenesis. Additionally, the invention can be applied to those at risk for developing a disease that responds to an inhibition of angiogenesis. The methods comprise administering to a mammal an effective angiogenesis-inhibiting amount of an matrilin-1 (including analogs, fragments or derivatives thereof having angiogenesis-inhibiting activity) in a composition substantially free of other cartilage protein.

As used herein, “substantially free of other cartilage protein” is meant to indicate that other cartilage proteins can be present in an incidental amount. In other words, the material is not intentionally added to an indicated composition, but may be present at a minor or inconsequential levels, for example, because it was carried over as an impurity as part of an intended composition component.

Any Matrilin-1, from any species, can be used in methods in the invention. It is further contemplated that other proteins with the antiangiogenic domains of Matrilin-1 (e.g. vWF domains, EGF-like domain, or N-glycosilation sites), such as Matrilin-2, Matrilin-3 and Matrilin-4, can be used in methods of the invention.

In one embodiment of the present invention, the Matrilin-1 is full length human Matrilin-1, amino acids 1-496 of SEQ ID NO.: 1. Alternatively, Matrilin-1 may be an angiogenesis-inhibiting fragment, analog, or derivative of SEQ ID NO.: 1. In one embodiment, Matrilin-1 is a peptide or peptides selected from the groups consisting of amino acids (aa's) 1-225, aa's 1-455, aa's 23-222, aa's 43-222, aa's 264-453, aa's 278-453. Such peptides can be linked together by peptide or other linkers or by using standard coupling chemistries.

In one embodiment of the present invention, the Matrilin-1 is full length shark Matrilin-1, or angiogenesis-inhibiting fragment, analog, or derivative thereof. Peptides derived from Shark Matrilin-1 can be linked together by peptide or other linkers or by using standard coupling chemistries.

In one embodiment of the present invention, the Matrilin-1 is full length chick Matrilin-1, amino acids 1-493 of SEQ ID NO.: 2. Alternatively, Matrilin-1 may be an angiogenesis-inhibiting fragment, analog, or derivative of SEQ ID NO. 2. In one embodiment, Matrilin-1 is a peptide or peptides selected from the groups consisting of amino acids (aa's) 1-220, aa's 1-450, aa's, 24-220, aa's 42-220, aa's 262-450, aa's 275-450. Such peptides can be linked together by peptide or other linkers or by using standard coupling chemistries.

In yet another embodiment, Matrilin comprises a fragment having at least 50% identity compared to a fragment of Matrilin from which the peptide was derived, wherein the fragment is derived from SEQ ID NO: 1, SEQ ID NO: 2, or the sequence of Shark Matrilin-1.

Furthermore, the present invention is directed to method of inhibiting angiogenesis in a tissue of a mammal having an angiogenic disease and/or cancer. The method comprises inhibiting angiogenisis in a tissue by delivering an effective angiogenesis-inhibiting amount of an matrilin-1 (including analogs, fragments or derivatives thereof having angiogenesis-inhibiting activity) in a composition substantially free of other cartilage protein to a mammal.

In another embodiment of the present invention, the methods are directed to the treatment of a solid tumor or solid tumor metastasis and said disease that responds to an inhibition of angiogenesis is cancer.

In another embodiment of the present invention, the methods are directed to the treatment of a blood borne or bone narrow derived tumors such as leukemia, multiple myeloma or lymphoma.

In yet another embodiment, the methods are directed to the treatment of retinal tissue and said disease that responds to an inhibition of angiogenesis is retinopathy, diabetic retinopathy, or macular degeneration.

In yet another embodiment, the methods of the present invention are directed toward treatment of atherosclerosis or a tissue at risk of restenosis, wherein the tissue is at the site of coronary angioplasty and said disease that responds to an inhibition of angiogenesis heart disease.

In another embodiment of the present invention, the methods are directed toward inhibiting angiogenesis in a tissue of a mammal, wherein said tissue is inflamed and said disease that responds to an inhibition of angiogenesis is arthritis (rheumatoid or osteo-arthritis).

The methods of the present invention can be used either alone, or in conjunction with other treatment methods known to those of skill in the art. Such methods may include, but are not limited to, chemotherapy, radiation therapy, or other known angiogenesis inhibitors.

In yet another embodiment of the present invention, said administering comprises intravenous, transdermal, intrasynovial, intramuscular, intraocular/periocular or oral administration. In another embodiment, the composition is formulated for sustained release. Alternatively, administration of the Matrilin-1 may comprise administering a gene therapy vector that constitutively or inducibly expresses full length Matrilin-1, a Matrilin derivative, or fragments thereof.

The methods of the present invention allow for the administration of Matrilin-1 either prophylactically or therapeutically.

The methods of the present invention are further directed toward treatment of a mammal at risk for developing a disease that responds to an inhibition of angiogenesis. The risk can be determined genetically. Alternatively, the risk can be determined by measuring levels of cancer marker proteins in the biological fluids (i.e. blood, urine) of a patient. Marker proteins include, for example, calcitonin, PSA, thymosin P-15, thymosin (3-16, and matrix metalloproteinases (MMPs).

In still another embodiment, the invention relates to the use of Matrilin-1 to prevent cell hyperproliferation and formation of clots along or around medical devices such as stents, catheters, cannulas, electrodes, and the like.

In one embodiment, Matrilin-1 may be systemically administered to a patient in which such a medical device has been inserted. In another embodiment, the medical device is coated with Matrilin-1 before insertion in the patient, and such Matrilin-1-coated medical devices are also envisaged by the present invention.

In yet another embodiment, a medical device comprising Matrilin-1 is provided. The medical device, for example, can be a stent, catheter, cannula, or an electrode.

A sustained release pharmaceutical composition that comprises Matrilin-1 is also provided.

### BRIEF DESCRIPTION OF FIGURES

FIGS. 1A and 1B show chromatography of concentrated cartilage extract (CCE).

FIG. 1A shows Biogel A-1.5M size-exclusion chromatography of CCE. Fractions 24-31 significantly inhibited EC proliferation. Fractions 52-75 possessed moderate TIMP activity, but had no significant effect on EC proliferation. These two sets of fractions were pooled separately (a and b), concentrated, dialyzed, and subjected to further purification (below). FIG. 1B shows Bio-Rex 70 cation-exchange chromatography of Pool a from Biogel A-1.5M. Fractions 2 and 5 displayed moderate inhibition of EC proliferation with no significant TIMP activity.

FIGS. 2A and 2B show inhibition of capillary EC growth and migration by MATN1 FIG. 2A shows inhibition of capillary EC growth by MATN1. Purified MATN1 was tested for its ability to inhibit βFGF-stimulated capillary EC and was found to inhibit EC proliferation in dose-dependent manner. FIG. 2B shows inhibition of capillary EC transwell migration by MATN1. Purified MATN1 was tested for its ability to inhibit βFGF-stimulated capillary EC and was found to inhibit EC migration in dose-dependent manner.

FIGS. 3A and 3B show inhibition of bFGF-stimulated capillary endothelial cell proliferation by recombinant chicken and human matrilin-1. Recombinant chicken (FIG. 3A) and human (FIG. 3B) MATN-1 were tested for there ability to inhibit bFGF-stimulated capillary endothelial cells as described in Examples I and II.

FIG. 4 shows a schematic of protein domain similarity between human Matrilin proteins. All matrilins have at least one von Willebrand type a factor, at least one EGF-like domain and a coiled-coil domain.

### DETAILED DESCRIPTION OF THE INVENTION

Matrilin-1 refers to compounds which are either native Matrilin-1, analogs of Matrilin-1, fragments of Matrilin (contiguous or noncontiguous) or synthetic peptides based partly on Matrilin-1 sequence. Matrilin-1 is also known as MATN1, or cartilage matrix protein (CMP). In mouse Matrilin-1 is known as CRTM. As used herein, Matrilin-1 refers to homologous sequences, for example Matrilin-1 from any species such as shark, human, chick, rat, or mouse.

The present invention relates generally to a method of treating a disease that responds to an inhibition of angiogenesis in a mammal. The method of the present invention comprises the administration of an effective amount of Matrilin-1 having antiangiogenic activity to a mammal.

Angiogenesis plays a role in a variety of disease processes. By inhibiting angiogenesis, one can intervene in the disease, ameliorate the symptoms, and in some cases cure the disease. Where the growth of new blood vessels is the cause of, or contributes to, the pathology associated with a disease, inhibition of angiogenesis will reduce the deleterious effects of the disease. Examples of diseases that respond to an inhibition of angiogenesis include, but are not limited to, rheumatoid arthritis, obesity, diabetic retinopathy, inflammatory diseases, restenosis, cancer, and the like. Where the growth of new blood vessels is required to support growth of a deleterious tissue, inhibition of angiogenesis will reduce the blood supply to the tissue and thereby contribute to reduction in tissue mass based on blood supply requirements. Examples include growth of tumors where neovascularization is a continual requirement in order that the tumor grows beyond a few millimeters in thickness, and for the establishment of solid tumor metastases.

One example of a disease responsive to inhibition of angiogenesis is ocular neovascular disease. This disease is characterized by invasion of new blood vessels into the structures of the eye such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization, and thus responsive to inhibition of angiogenisis include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, rheumatoid arthritis, systemic lupus, polyarteritis, trauma, Wegener's sarcoidosis, scleritis, Stevens-Johnson disease, pemnphigoid, radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy.

Another disease responsive to inhibition of angiogenesis is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogenic-related factors contributes to the destruction of the joint. At a later stage, the angiogenic factors would promote new bone formation. Therapeutic intervention that prevents the bone destruction could halt the progress of the disease and provide relief for persons suffering with arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity. Inhibitors of angiogenesis could be useful to prevent atherosclerosis progression or plaque restenosis after angioplasty.

One of the most frequent angiogenic diseases of childhood is the hemangioma, a vascular anomaly. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use. The present invention is useful for treatment of hemangiomas and other vascular anomalies.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula. In addition, dysregulated angiogenesis is responsible for Klippel-Trenaunay syndrome which is characterized by malformations of capillary, venous, and lymphatic vessels; and by bony and soft tissue hypertrophy.

Angiogenesis is prominent in solid tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors (prostate, breast, lung, colon, uterine, skin, ovarian . . . ) and benign tumors such as acoustic neuroma, neurofibroma, trachoma and pyogenic granulomas.

It should be noted that angiogenesis has been associated with blood-born tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors and other diseases such as multiple myeloma and lymphoma.

Angiogenesis is important in two stages of tumor metastasis. The first stage where angiogenesis stimulation is important is in the vascularization of the tumor which allows tumor cells to enter the blood stream and to circulate throughout the body. After the tumor cells have left the primary site, and have settled into the secondary, metastasis site, angiogenesis must occur before the new tumor can grow and expand. Therefore, prevention of angiogenesis could lead to the prevention of metastasis of tumors and possibly contain the neoplastic growth at the primary site.

Knowledge of the role of angiogenesis in the maintenance and metastasis of tumors has led to a prognostic indicator for breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by therapeutic means could possibly lead to cessation of the recurrence of the tumors.

Angiogenesis is also involved in normal physiological processes such as reproduction and wound healing. Angiogenesis is an important step in ovulation, endometrial proliferation and also in implantation of the blastula after fertilization. Prevention of angiogenesis could be used to induce amenorrhea, to block ovulation, to prevent implantation by the blastula and to inhibit endometriosis. Angiogenesis is also involved in other normal physiological processes such as fat accumulation and expansion. Thus angiogenesis inhibition is useful to treat obesity.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

The invention provides for a method for inhibiting a disease that responds to an inhibition of angiogenesis through the inhibition of angiogenesis in a tissue using Matrilin-1, or angiogenesis inhibiting fragment thereof. Matrilin-1, or angiogenesis inhibiting fragment thereof inhibits events in a tissue which depend upon angiogenesis and thereby inhibits a disease that responds to an inhibition of angiogenesis. Generally, the method comprises administering to the tissue a composition comprising an angiogenesis-inhibiting amount of Matrilin-1. In one embodiment of the present invention, Matrilin-1 comprises the full length protein, herein described as SEQ ID NO.: 1 or SEQ ID NO: 2. Alternatively, Matrilin-1 may be an angiogenesis-inhibiting fragment, analog, or derivative of SEQ ID NO.: 1, SEQ ID NO: 2, or of the amino acid sequence of Shark Matrilin-1. It is also contemplated that dimeric and trimeric forms of Matrilin-1 can be used.

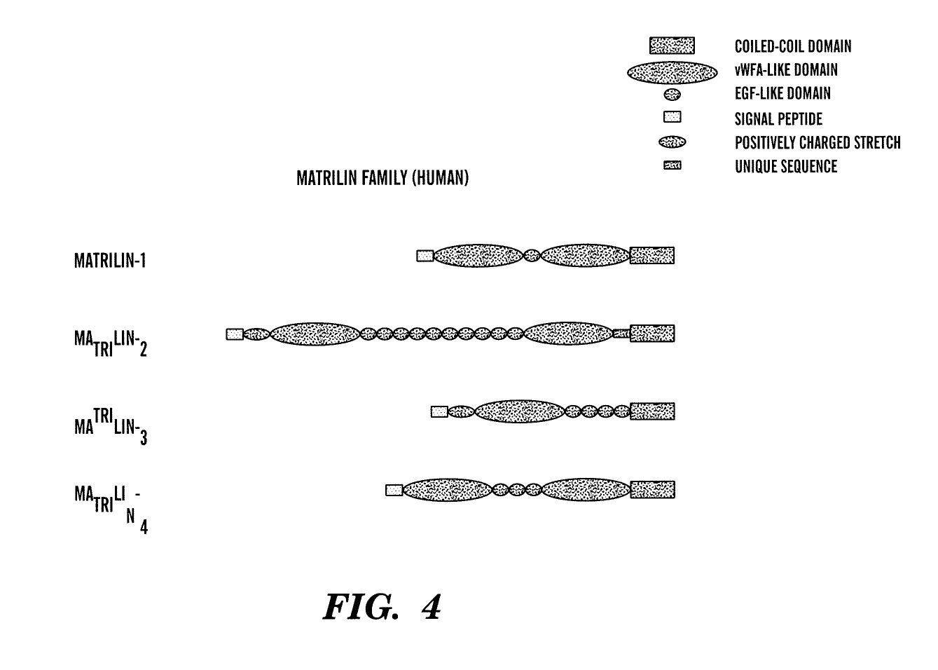

It is further contemplated that other proteins, or fragments thereof, with similar domains as Matrilin-1 (e.g. vWF domains, EGF-like domain, or N-glycosilation sites), such as Matrilin-2, Matrilin-3 and Matrilin-4, can be used in methods of the invention (See FIG. 4). The genebank accessions for human Matrilin-2 is NP_{—}002371 (SEQ ID NO: 3); human Matrilin-3 is CAA12110 (SEQ ID NO: 4); Human Matrilin-4 is CAA07569 (SEQ ID NO: 5).

Matrilin-1 (MATN-1), a member of the oligomeric matrilin family of proteins (Deak et al., 1999), is a non-collagenous, multiadhesion protein (Rentsendorj et al., 2005) containing two ‘sticky’ von Willebrand Factor A-like (vWF) domains that compose approximately 78.6% and 78.3% of the amino acid composition of human and chicken proteins, respectively. The adhesion of vWFA-like domains is mediated by metal ion-dependent adhesion site (MIDAS) motifs approximately 176 to 180 amino acids in length (Chen et al., 1999). The vWFA-like domains of MATN-1 have been found to bind type II collagen and aggrecan (Deak et al., 1999), biglycan or decorin (Wiberg et al., 2003), as well as self-binding (Chen et al., 1999) and binding with Matrilin-3 to form hetero-trimers and hetero-dimers (Zhang and Chen, 2000).

Matrilin-1 is known to interact and bind with the α1β1 integrin (Makihara et al., 1999), a known regulator of angiogenic promoter, VEGF (Senger et al., 1997). Matrilin-1 may possibly act like a disintegrin (an anti-adhesive protein), which blocks mediation of the integrins by competitively binding to them.

Matrilin-1 proteins or peptides, fragment, analog or derivative useful in the treatment of angiogenic diseases as described in the present invention will inhibit angiogenesis in the corneal neovascularization assay (Gimbrone, M A. et al. (1974) J Natl Canc Inst. 52:413-427; Kenyon, B M. et al. (1996) Invest Opthalmol V is Sci 37:1625-1632; Kenyon, B M. et al. (1997) Exp Eye Res 64:971-97; Proia, A D. et al. (1993) Exp Eye Res 57:693-698) by at least 25%, more preferably, by at least 50%.

In one preferred embodiment, Matrilin-1 comprises amino acids selected from amino acids (AA's) 1-225, aa's 1-455, aa's 23-222, aa's 43-222, aa's 264-453, or aa's 278-453 of SEQ ID NO.: 1. Peptides, analogs, or derivatives thereof derived from SEQ ID NO: 1, or SEQ ID NO: 2, can be linked together by peptide or other linkers or by using standard coupling chemistries. Such fragments (peptides) can be at least 8, 10, 20, 30, 40, 50, 75, 100, or 150 amino acids in length.

In one embodiment, Matrilin-1 comprises a derivative of SEQ ID NO: 1, SEQ ID NO: 2, or the amino acid sequence of Shark Matrilin-1, having at least 50% identity compared to a fragment of Matrilin-1 from which the derivative was derived.

### Angiogenesis Screening Assays

Examples of well described angiogenesis screening assays that may be initially used to test the antiangiogenic activity of Matrilin-1 include, but are not limited to, in vitro endothelial cell assays, rat aortic ring angiogenesis assays, cornea micropocket assays (corneal neovascularization assays), and chick embryo chorioallantoic membrane assays (Erwin, A. et al. (2001) Seminars in Oncology 28(6):570-576).

Some example in vitro endothelial cell assays include methods for monitoring endothelial cell proliferation, cell migration, or tube formation. Cell proliferation assays may use cell counting, BRdU incorporation, thymidine incorporation, or staining techniques (Montesano, R. (1992) Eur J Clin Invest 22:504-515; Montesano, R. (1986) Proc Natl. Acad. Sci. USA 83:7297-7301; Holmgren L. et al. (1995) Nature Med 1:149-153).

In the cell migration assays endothelial cells are plated on matrigel and migration monitored upon addition of a chemoattractant (Homgren, L. et al. (1995) Nature Med 1:149-153; Albini, A. et al. (1987) Cancer Res. 47:3239-3245; Hu, G. et al. (1994) Proc Natl Acad Sci USA 6:12096-12100; Alessandri, G. et al. (1983) Cancer Res. 43:1790-1797.)

The endothelial tube formation assays monitor vessel formation (Kohn, E C. et al. (1995) Proc Natl Acad Sci USA 92:1307-1311; Schnaper, H W. et al. (1995) J Cell Physiol 165:107-118).

Rat aortic ring assays have been used successfully for the screening of angiogenesis drugs (Zhu, W H. et al. (2000) Lab Invest 80:545-555; Kruger, E A. et al. (2000) Invasion Metastas 18:209-218; Kruger, E A. et al. (2000) Biochem Biophys Res Commun 268:183-191; Bauer, K S. et al. (1998) Biochem Pharmacol 55:1827-1834; Bauer, K S. et al. (2000) J Pharmacol Exp Ther 292:31-37; Berger, A C. et al. (2000) Microvasc Res 60:70-80.). Briefly, the assay is an ex vivo model of explant rat aortic ring cultures in a three dimensional matrix. One can visually observe either the presence or absence of microvessel outgrowths. The human saphenous angiogenesis assay, another ex vivo assay, may also be used (Kruger, E A. et al. (2000) Biochem Biophys Res Commun 268:183-191).

Another common screening assay is the cornea micropocket assay (Gimbrone, M A. et al. (1974) J Natl Canc Inst. 52:413-427; Kenyon, B M. et al. (1996) Invest Opthalmol V is Sci 37:1625-1632; Kenyon, B M. et al. (1997) Exp Eye Res 64:971-978; Proia, A D. et al. (1993) Exp Eye Res 57:693-698). Briefly, neovascularization into an avascular space is monitored in vivo. This assay is commonly performed in rabbit, rat, or mouse.

The chick embryo chorioallantoic membrane assay has been used often to study tumor angiogenesis, angiogenic factors, and antiangiogenic compounds (Knighton, D. et al. (1977) Br J Cancer 35:347-356; Auerbach, R. et al. (1974) Dev Biol 41:391-394; Ausprunk, D H. et al. (1974) Dev Biol 38:237-248; Nguyen, M. et al. (1994) Microvasc Res 47:31-40). This assay uses fertilized eggs and monitors the formation of primitive blood vessels that form in the allantois, an extra-embryonic membrane.

The above is just a sampling of angiogenic inhibitor assays that may be used to assess the antiangiogenic activity of Matrilin-1.

### Cancer Screening Assays:

### Mouse Models to Study Anticancer Properties of Matrilin-1

Lewis lung carcinoma is one commonly used tumor in mice to study inhibitors of cancer. The tumor is maintained by passage from animal to animal. Mice with Lewis lung carcinomas of 600-1200 mm³ tumors are sacrificed and the skin overlying the tumor cleaned with betadine and ethanol. In a laminar flow hood, tumor tissue is excised under aseptic conditions. A suspension of tumor cells in 0.9% normal saline is made by passage of viable tumor tissue through a sieve and a series of sequentially smaller hypodermic needles of diameter 22- to 30-gauge. The final concentration is adjusted to 1×10⁷ cells/ml and the suspension is placed on ice. After the site is cleaned with ethanol, the subcutaneous dorsa of mice in the proximal midline are injected with 1×10⁶ cells in 0.1 ml of saline.

To detect inhibition with Matrilin-1, mice can be implanted with Lewis lung carcinomas as described above. Tumors are measured with a dial-caliper and tumor volumes were determined using the formula width 2×length×0.52, and the ratio of treated to control tumor volume (T/C) was determined for the last time point. After tumor volume was 100-200 mm³ (0.5-1% of body weight), which occurs within 3-7 days, mice are randomized into two groups. One group receives Matrilin-1 injected intraperitoneal once daily. The other group receives comparable injections of the vehicle alone. The experiments are terminated and mice are sacrificed and autopsied when the control mice began to die.

The gene encoding human Matrilin-1 has been sequenced. The human sequence has been assigned genebank accession number NM_{—}002379. Mouse Matrilin-1 (NM_{—}010769), Rat Matrilin-1 (NM_{—}001006976), and Canine Matrilin-1 (XM_{—}54451) have also been cloned. Matrilin-1 can be isolated from its natural source or it can be produced by recombinant means, or by chemical synthesis.

As described earlier, angiogenesis includes a variety of processes involving neovascularization of a tissue including “sprouting”, vasculogenesis, or vessel enlargement. With the exception of traumatic wound healing, corpus leuteum formation and embryogenesis, it is believed that the majority of angiogenesis processes are associated with disease processes and therefore the use of the present therapeutic methods are selective for the disease and do not have deleterious side effects.

There are a variety of “diseases that respond to an inhibition of angiogenesis” also, referred to as “angiogenic diseases/disorders” including, but not limited to, obesity, inflammatory disorders such as immune and non-immune inflammation, chronic articular rheumatism and psoriasis, endometriosis, disorders associated with inappropriate or inopportune invasion of vessels such as diabetic retinopathy, macular degeneration, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli.

The patient treated in the present invention in its many embodiments is desirably a human patient, although it is to be understood that the principles of the invention indicate that the invention is effective with respect to all mammals, which are intended to be included in the term “patient”. In this context, a mammal is understood to include any mammalian species in which treatment of diseases associated with angiogenesis is desirable, particularly agricultural and domestic mammalian species.

Thus, in one related embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In another related embodiment, a tissue to be treated is a retinal tissue of a patient with a retinal disease such as diabetic retinopathy, macular degeneration or neovascular glaucoma and the angiogenesis to be inhibited is retinal tissue angiogenesis where there is neovascularization of retinal tissue.

In an additional related embodiment, a tissue to be treated is a tumor tissue of a patient with a solid tumor, metastases, a skin cancer, a breast cancer, a medullary thyroid cancer, a hemangioma or anigiofibroma and the like cancer, and the angiogenesis to be inhibited is tumor tissue angiogenesis where there is neovascularization of a tumor tissue. Tumors which may be prevented or inhibited by preventing or inhibiting angiogenesis with the present invention include, but are not limited to lung tumors, pancreas tumors, breast tumors, colon tumors, laryngeal tumors, ovarian tumors, thyroid tumors, melanoma, adenocarcinoma, sarcomas, thymoma, lymphoma, liver tumors, kidney tumors, non-Hodgkins lymphoma, Hodgkins lymphoma, leukemias, uterine tumors, prostate tumors, renal tumors, brain tumors, testicular tumors, bone tumors, muscle tumors, tumors of the placenta, gastric tumors and the like. The diseases listed above are all diseases responsive to inhibition of angiogenisis.

Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the practice of the method in conjunction with other therapies such as conventional chemotherapy or radiation therapy directed against solid tumors and for control of establishment of metastases. The administration of angiogenesis-inhibiting amounts of Matrilin-1 may be conducted before, during or after chemotherapy or radiation therapy. In addition, the compounds of the present invention may be administered concurrently with other cancer therapies known to those of skill in the art. For example, Matrilin-1 may be combined with chemotherapy, radiation, or other known angiogenesis inhibitors. Known angiogenesis inhibitors include, but are not limited to: Angiostatin, Bevacizuniab (Avastin), Arresten, Canstatin, Combretastatin, Endostatin, NM-3, Thrombospondin, Tumstatin, 2-methoxyestradiol, Vitaxin, ZD1839 (Iressa), ZD6474, OSI774 (Tarceva), CI1033, PKI1666, IMC225 (Erbitux), PTK787, SU6668, SU11248, Herceptin, and IFN-α, CELEBREX® (Celecoxib), THALOMID® (Thalidomide), Caplostatin (WO/A2005103281) and IFN-α (Kerbel et al., Nature Reviews, Vol. 2, October 2002, pp. 727). For combination therapy, the dose of Matrilin-1 may be administered prior to, concurrently, or after administration of a second anti-angiogenic agent or chemotherapeutic agent. Furthermore, the Matrilin-1 may be administered alone or in combination with another anti-angiogenic compound prior to, concurrently, or after the surgical removal of a solid tumor mass.

In the method of treatment, the administration of Matrilin-1 may be for either “prophylactic” or “therapeutic” purpose. When provided prophylactically, Matrilin-1 is provided in advance of any symptom. The prophylactic administration of the Matrilin-1 serves to prevent or inhibit an angiogenesis disease or disorder, i.e. cancer. Prophylactic administration of Matrilin-1 may be given to a patient with, for example, a family history of cancer. Alternatively, administration of Matrilin-1 may be given to a patient with rising cancer marker protein levels. Such markers include, for example, rising PSA, thymosin β-15, thymosin β-16, calcitonin, matrix metalloproteinase (MMP), and myeloma M-protein.

When provided therapeutically, Matrilin-1 is provided at (or after) the onset of a symptom or indication of angiogenesis. Thus, Matrilin-1 may be provided either prior to the anticipated angiogenesis at a site or after the angiogenesis has begun at a site.

Insofar as the present methods apply to inhibition of tumor neovascularization, the methods can also apply to inhibition of tumor tissue growth, to inhibition of tumor metastases formation, and to regression of established tumors.

Restenosis is a process of smooth muscle cell (SMC) migration and proliferation at the site of percutaneous transluminal coronary angioplasty which hampers the success of angioplasty. The migration and proliferation of SMC's during restenosis can be considered a process of angiogenesis which is inhibited by the present methods. Therefore, the invention also contemplates inhibition of restenosis by inhibiting angiogenesis according to the present methods in a patient following angioplasty procedures. For inhibition of restenosis, an angiogenesis-inhibiting amount of Matrilin-1 is typically administered after the angioplasty. The administration of the compounds of the invention may occur from about 2 to about 28 days post-angioplasty and more typically for about the first 14 days following the procedure.

The present method for inhibiting angiogenesis in a tissue, and therefore for also practicing the methods for treatment of angiogenesis-related diseases, comprises contacting a tissue in which angiogenesis is occurring, or is at risk for occurring, with a composition comprising a therapeutically effective amount of Matrilin-1. Thus the method comprises administering to a patient a therapeutically effective amount of a physiologically tolerable composition containing Matrilin-1 of the invention.

The effective dosage range for the administration of Matrilin-1 depends upon the form of Matrilin-1, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of Matrilin-1 sufficient to produce a measurable inhibition of angiogenesis or tumor growth in the tissue being treated, i.e., an angiogenesis-inhibiting amount. Inhibition of angiogenesis can be measured in situ by immunohistochemistry, or by other methods known to one skilled in the art.

One skilled in the art can readily assess the potency of a candidate Matrilin-1 of this invention.

In general, it is desirable to provide the recipient with a dosage of Matrilin-1 of at least about 10 μg/kg, preferably at least about 10 mg/kg or higher. A range of from about 1 μg/kg to about 100 mg/kg is preferred although a lower or higher dose may be administered. The dose provides an effective antiangiogenic serum or tissue level of Matrilin-1. The dose is administered at least once and may be provided as a bolus, a continuous administration or sustained release. Multiple administration over a period of weeks or months may be preferable. It may also be preferable to administer Matrilin-1 at least once/week and even more frequent administrations (e.g. daily). Subsequent doses may be administered as indicated.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The compounds of the invention can be administered parenterally by injection or by gradual infusion over time and can be delivered by peristaltic means.

This invention may also be used on a stent or other medical device to prevent angiogenesis and restenosis in the tissue in which it is implanted.

Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics.

For topical administration, Matrilin-1 is formulated into ointments, salves, gels, or creams, as is generally known in the art.

The therapeutic compositions of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual.

### Therapeutic Compositions

The Matrilin-1 useful for practicing the methods of the present invention are described herein. Any formulation or drug delivery system containing the active ingredients, which is suitable for the intended use, as are generally known to those of skill in the art, can be used. Suitable pharmaceutically acceptable carriers for oral, rectal, topical or parenteral (including inhaled, subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

As antiangiogenic agents typically need to be administered over a period of time, in certain embodiments the Matrilin-1 is formulated as a sustained release composition. For example, sustained-release pharmaceutical compositions include, but are not limited to, sustained-release matrices such as biodegradable matrices or semi-permeable polymer matrices in the form of shaped articles, e.g., films, or mirocapsules that comprise Matrilin-1.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

Sustained-release matrices include polylactides (U.S. Pat. No. 3,773,919, EP 58,481), copolymners of L-glutamic acid and gamma-ethyl-L-glutamate (U. Sidman et al., Biopolymers 22:547-556 (1983)), poly (2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release Matrilin compositions also include liposomally entrapped Matrilin. Liposomes containing Matrilin are prepared by methods known per se: DE 3,218,121; Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Matrilin-1 therapy. Other biodegradable polymers and their use are described, for example, in detail in Brem et al. (1991, J. Neurosurg. 74:441-446).

In one embodiment, osmotic minipumps are used to provide controlled sustained delivery of Matrilin-1 anti-angiogenic protein, or fragment thereof, through cannulae to the site of interest, e.g. directly into a tissue at the site of metastatic growth or into the vascular supply of a tumor. The pump can be surgically implanted, for example continuous administration of endostatin, an anti-angiogenesis agent, by intraperitoneally implanted osmotic pump is described in Cancer Res. 2001 Oct. 15; 61 (20):7669-74. Matrilin-1 can also be continually administered by a external pump attached to an intravenous needle.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compound which upon dilution with an appropriate solvent give a solution suitable for parental administration above.

For enteral administration, a compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylamino-1-propanesulfonate), Big-CHAPS(N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

Alternatively, the compound may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. A review of known methods is provided by G. Gregoriadis, Chapter 14, “Liposomes,” Drug Carriers in Biology and Medicine, pp. 287-341 (Academic Press, 1979).

Microspheres formed of polymers or proteins are well known to those skilled in the art, and can Se tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS19:155-157 (1998), the contents of which are hereby incorporated by reference.

In one embodiment, Matrilin-1 can be formulated into a liposome or microparticle which is suitably sized to lodge in capillary beds following intravenous administration. When the liposome or microparticle is lodged in the capillary beds surrounding ischemic tissue, the agents can be administered locally to the site at which they can be most effective. Suitable liposomes for targeting ischemic tissue are generally less than about 200 nanometers and are also typically unilamellar vesicles, as disclosed, for example, in U.S. Pat. No. 5,593,688 to Baldeschweiler, entitled “Liposomal targeting of ischemic tissue,” the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

The formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

Matrilin-1 may be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, more preferably between 2 and 5 microns.

Generally for nasal administration a mildly acid pH will be preferred. Preferably the compositions of the invention have a pH of from about 3 to 5, more preferably from about 3.5 to about 3.9 and most preferably 3.7. Adjustment of the pH is achieved by addition of an appropriate acid, such as hydrochloric acid.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

Matrilin-1 of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

A therapeutic composition contains an angiogenesis-inhibiting amount of Matrilin-1 of the present invention.

### Polypeptides

It should be understood that a subject the polypeptide Matrilin-1 need not be identical to the amino acid sequence of human Matrilin-1 (SEQ ID. NO 1) (or shark or chick Matrilin-1), so long as it has 50% identity to the derivative of Matrilin-1 from which it was derived and has angiogenesis inhibiting activity. In another embodiment, the derivative of Matrilin-1 has at least 75% identity to the Matrilin-1 derivative from which it was derived. In a most preferred embodiment, the derivative of Matrilin-1 has at least 90% identity to the Matrilin-1 from which it was derived. As described previously, Matrilin-1 can also be Matrilin-1 from another species, such as shark, chick or mouse. Preferably, Matrilin-1 is human or shark Matrilin-1.

A subject Matrilin-1 includes any analog, fragment or chemical derivative of a polypeptide whose amino acid residue sequence is shown herein so long as the polypeptide is angiogenesis-inhibiting or cancer-inhibiting. Therefore, a present polypeptide can be subject to various changes, substitutions, insertions, and deletions where such changes provide for certain advantages in its use. In this regard, the Matrilin-1 of this invention corresponds to, rather than is identical to, the sequence of a recited peptide where one or more changes are made and it retains the ability to function as an angiogenesis inhibitor in one or more of the assays as defined herein. The use of Matrilin-2, Matrilin-3, Matrilin-4, and angiogenic inhibiting fragments thereof, are also contemplated.

Thus, a Matrilin-1 can be in any of a variety of forms of peptide derivatives, which include amides, conjugates with proteins, cyclic peptides, polymerized peptides, analogs, fragments, chemically modified peptides, and the like derivatives.

The term “analog” includes any polypeptide having an amino acid residue sequence substantially identical to a sequence specifically shown herein in which one or more residues have been conservatively substituted with a functionally similar residue and which displays angiogenesis-inhibiting activity as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such polypeptide displays the requisite inhibition activity.

A “chemical derivative” refers to a subject polypeptide having one or more residues chemically derivatized by reaction of a functional side group. In addition to side group derivations, a chemical derivative can have one or more backbone modifications including alpha-amino substitutions such as N-methyl, N-ethyl, N-propyl and the like, and alpha-carbonyl substitutions such as thioester, thioamide, guanidino and the like. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to from salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. Also included as chemical derivatives are those peptides which contain one or more commonly available, non-natural amino acids. For example those available for peptide synthesis from commercial suppliers (e.g. Bachem Catalog, 2004 pp. 1-276). For examples: 4-hydroxyproline may be substituted for proline; 5-hydroxylysine may be substituted for lysine; 3-methylhistidine may be substituted for histidine; homoserine may be substituted for serine; ornithine may be substituted for lysine; β-alanine may be substituted for alanine; norleucine may be substituted for leucine; phenylglycine may be substituted for phenylalanine, and L-1,2,3,4-tetrahydronorharman-3-carboxylic acid or H-β-(3-Benzothienyl)-Ala-OH may be substituted for tryptophan. Polypeptides of the present invention also include any polypeptide having one or more additions and/or deletions or residues relative to the sequence of a polypeptide whose sequence is shown herein, so long as the requisite activity is maintained.

As with all therapies involving proteins and peptides, reducing immunogenicity and prolonging half-life may be necessary to enhance the efficacy (Hermeling Pharm Res. 2004 June; 21(6):897-903.). Such methods to reduce immunogenicity are numerous including such well known examples as the conjugation of the protein with polyalkylene glycols (such as polyethylene glycol/PEG and polyethylene oxide), the alteration of amino acids to reduce potential T cell epitopes, co-administration with immunosuppressive drugs and production of fusion proteins (such as Fc antibody fragments fusion proteins).

The term “fragment” refers to any subject polypeptide having an amino acid residue sequence shorter than that of a polypeptide whose amino acid residue sequence is shown herein.

When a polypeptide of the present invention has a sequence that is not identical to the sequence of Matrilin-1, it is typically because one or more conservative or non-conservative substitutions have been made, usually no more than about 30 percent, and preferably no more than 10 percent of the amino acid residues are substituted. Additional residues may also be added at either terminus of a polypeptide for the purpose of providing a “linker” by which the polypeptides of this invention can be conveniently affixed to a label or solid matrix, or carrier.

Labels, solid matrices and carriers that can be used with the polypeptides of this invention are described herein.

Amino acid residue linkers are usually at least one residue and can be 40 or more residues, more often 1 to 10 residues and may couple polypeptides or proteins covalently or non-covalently. Typical amino acid residues used for linking are glycine, tyrosine, cysteine, lysine, glutamic and aspartic acid, or the like. In addition, a subject polypeptide can differ, unless otherwise specified, from the natural sequence of Matrilin-1 by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present.

Peptide sequences of the present invention may also be linked together using non-peptide crosslinkers (Pierce 2003-2004 Applications Handbook and Catalog, Chapter 6) or other scaffolds such as HPMA, polydextran, polysaccharides, ethylene-glycol, poly-ethylene-glycol, glycerol, sugars, and sugar alcohols (e.g. sorbitol, mannitol). Such linked peptide may be composed of one or more, identical or different sequences or subsequences of Matrilin-1.

Any peptide of the present invention may be used in the form of a pharmaceutically acceptable salt. Suitable acids which are capable of forming salts with the peptides of the present invention include inorganic acids such as trifluoroacetic acid (TFA), trichloroacetic acid (TCA), hydrochloric acid (HCl), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

In addition, Matrilin-1 can be provided in the form of a fusion protein. Fusion proteins are proteins produced by recombinant DNA methods as described herein in which the subject polypeptide is expressed as a fusion with a second carrier protein such as a glutathione sulfhydryl transferase (GST) or other well known carrier.

In one preferred embodiment, Matrilin-1 fragments, analogs, or derivatives thereof are linked together via a peptide or other linker. A “peptide linker” is a short (e.g., about 1-40, e.g., 1-20 amino acids) sequence of amino acids that is not part of the sequence of either of two polypeptides being joined. A linker peptide is attached on its amino-terminal end to one polypeptide or polypeptide domain and on its carboxyl-terminal end to another polypeptide or polypeptide domain. Examples of useful linker peptides include, but are not limited to, glycine polymers ((G)n) including glycine-serine and glycine-alanine polymers (e.g., a (Gly4Ser)n repeat where n=1-8, preferably, n=3, 4, 5, or 6). Matrilin-1 fragments, analogs, or derivatives thereof can also be joined by chemical bond linkages, such as linkages by disulfide bonds or by chemical bridges.

### Gene Therapy

The Matrilin-1 of the present invention may be administered to a patient by any one of several gene therapy techniques known to those of skill in the art. In general, gene therapy can be accomplished by either direct transformation of target cells within the mammalian subject (in vivo gene therapy) or transformation of cells in vitro and subsequent implantation of the transformed cells into the mammalian subject (ex vivo gene therapy).

U.S. Pat. No. 6,531,456 provides methods for the successful transfer of a gene into a solid tumor cell using recombinant AAV virions. Generally, the method described in U.S. Pat. No. 6,531,456 allows for the direct, in vivo injection of recombinant AAV virions into tumor cell masses, e.g., by intra-tumoral injection. The invention also provides for the simultaneous delivery of a second gene using the recombinant AAV virions, wherein the second gene is capable of providing an ancillary therapeutic effect when expressed within the transduced cell.

The recombinant AAV virions described above, including the DNA of interest, can be produced using standard methodology, known to those of skill in the art. The methods generally involve the steps of (1) introducing an AAV vector into a host cell; (2) introducing an AAV helper construct into the host cell, where the helper construct includes AAV coding regions capable of being expressed in the host cell to complement AAV helper functions missing from the AAV vector; (3) introducing one or more helper viruses and/or accessory function vectors into the host cell, wherein the helper virus and/or accessory function vectors provide accessory functions capable of supporting efficient recombinant AAV (“rAAV”) virion production in the host cell; and (4) culturing the host cell to produce rAAV-virions. The AAV vector, AAV helper construct and the helper virus or accessory function vector(s) can be introduced into the host cell either simultaneously or serially, using standard transfection techniques.

Matrilin-1s used in the methods of the present invention can be delivered systemically via in vivo gene therapy. Systemic treatment involves transfecting target cells with the DNA of interest, i.e. Matrilin-1 or Matrilin-1 fragments, analogs, or derivatives thereof, expressing the coded protein in that cell, and the capability of the transformed cell to subsequently secrete the manufactured protein into blood.

A variety of methods have been developed to accomplish in vivo transformation including mechanical means (e.g, direct injection of nucleic acid into target cells or particle bombardment), recombinant viruses, liposomes, and receptor-mediated enidocytosis (RME) (for reviews, see Chang et al. 1994 Gastroenterol. 106:1076-84; Morsy et al. 1993 JAMA 270:2338-45; and Ledley 1992 J. Pediatr. Gastroenterol. Nutr. 14:328-37).

### EXAMPLES

### Example I

Experimental Procedures

Extract Preparation—Spiny dogfish (Squalus acanthias) were collected in July 2003. Two hundred fifty g of cleaned cartilage was homogenized and extracted in 4 L of a 2 M NaCl, 0.01M HEPES, 3 mM EDTA, 0.02% NaN₃ extraction buffer for 4 days under constant agitation. The extraction solution was filtered with gauze, centrifuged at 6,500×g for 2 hours to remove particulates, then concentrated with a Vivacell 250 (10,000 MWCO) to a final concentration of approximately 5 ml. The concentrated cartilage extract (CCE) had a final protein concentration of 7.8 mg/ml. All procedures were performed at 4° C.

Angiogenesis Antibody Arrays—Angiogenesis antibody arrays allowed for the simultaneous detection of up to 20 different angiogenic modulators. 500 μg of CCE was added to RayBio Human Angiogenesis Antibody Array 1 (Raybiotech, Inc., Norcross, Ga.; H0118001A) and 2 mg of CCE was added to the Transignal Angiogenesis Antibody Array (Panomics, Inc., Redwood City, Calif.; MA6310) according to the manufacturer instructions.

SDS-PA GE Electrophoresis and Western Blot Analysis—Proteins were resolved on 12% SDS-PAGE or NU-PAGE gels (Invitrogen) run at 125 V or 200 V for 1 hr and visualized by either silver or SYPRO Ruby (Molecular Probes, ) staining. All other potential angiogenic regulators were screened for using traditional SDS-PAGE electrophoresis follow by Western Blot onto nitrocellulose membrane. Antibodies to known angiogenesis inhibitors not found in the antibody arrays were used to probe for IL-12 (interleukin 12), IP-10 (interferon-inducible protein 10), and TIMP-1 (tissue inhibitor of metalloproteinases 1). HRP-conjugated anti-mouse or anti-rabbit (sources) was applied onto the membrane as a secondary antibody. The signals were detected with the SuperSignal West Pico Chemiluminescent Substrate (Pierce, Rockford, Ill.) according to the manufacturer's instructions.

Partial Purification of inhibitors of a Angiogenesis from CCE-CCE was fractionated using size-exclusion and ion-exchange chromatography. For size exclusion chromatography, CCE was prepared by dialyzing against Biogel A-1.5M buffer (4.0M Guanidine HCl, 0.02M NaCl, 0.001M CaCl₂, 0.02% NaN₃, pH 7.6). The prepared sample was applied to a Biogel A-1.5M size exclusion column (2.5×45 cm) after column was calibrated with gel filtration standards (Bio-Rad #151-1901) [Thyroglobulin (670 kDa), γ-globulin (158 kDa), Ovalbumin (44 kDa), myoglobin (17 kDa), and vitamin B₁₂ (1.35 kDa)], and washed with 3 volumes of buffer. Every third fraction off the column was assayed for inhibition of EC proliferation and TIMP activity (see protocol below). Samples of interest that inhibited either proliferation or MMP activity, or both were pooled, dialyzed against Bio-Rex buffer (0.05M Tris, 0.05M NaCl, 0.001M CaCl₂, 0.02% NaN₃, pH 7.6), and concentrated to a volume of 1 ml. The prepared samples were applied to a Bio-Rex 70 (2.5×10 cm), which had been previously calibrated in Bio-Rex buffer. The NaCl concentration was increased isocratically from 0.05M to 1.0M.

Identification of protein candidates—Selected anti-proliferation fractions off size-exclusion or cation-exchange columns were subjected to reducing, denaturing electrophoresis on 12% SDS-PAGE gels (BioRad) or 12% Bis-Tris NU-PAGE gels (Invitrogen). Selected proteins were excised from the gel with a razor blade and sent out for MS/MS (Dana Farber Facility, Boston, Mass.).

MMP Inhibitory Activity—MMP inhibitory activity was assessed using a quantitative [14^{C}]collagen film assay, as described previously Moses et al. (1990). Carbon 14-labeled collagen was added to 96-well plates and allowed to polymerize. To determine inhibitory activity, wells were treated with a known amount of activated type I collagenase plus test sample or with collagenase alone and the plates incubated at 37° C. for 2.5 h to allow for release of 14^{C} by the enzyme. Superantants were then analyzed in a Wallac scintillation counter, and percent inhibition of collagenolytic activity was calculated. An IC₅₀ unit was defined as the amount of protein necessary to inhibit the proteolytic activity of collagenase by 50%.

Cell Culture-Capillary endothelial cells (EC), isolated from bovine adrenal cortex, were a kind gift of Dr. Judah Folkman (Children's Hospital, Boston). Cells were maintained in Dulbecco's modified Eagle's medium (DMEM; Invitrogen) containing 10% calf serum (HyClone) and 3 ng/ml basic fibroblast growth factor (bFGF). Cells were maintained in an atmosphere of 10% CO₂ at 37° C.

Capillary Endothelial Cell Proliferation—Capillary EC proliferation was measured as reported previously by Moses et al., (1990, 1992, 1999). Briefly, capillary EC were plated on pregelatinized 96-well plates at a density of 2000 cells per well in Dulbecco's modified Eagle's medium supplemented with 5% calf serum and allowed to attach for 24 h. The next day, cells were treated with fresh medium with or without 1 ng/ml bFGF and challenged with the test proteins at various concentrations. All samples were tested in duplicate. Control wells contained cell treated with medium alone or medium with bFGF. After 72 h, the medium was removed, and the cells were lysed in buffer containing Triton X-100 and the phosphates substrate p-nitrophenyl phosphate. After a 2 h incubation at 37° C., NaOH was added to each well to terminate the reaction and cell density was determined by calorimetric analysis using a SpectraMax 190 multiwell plate reader (Molecular Devices, Sunnyvale, Calif.). All samples were tested in duplicate in at least three independent experiments.

Capillary Endothelial Cell Migration—The motility response of capillary EC was assayed using a modified Boyden chamber. The upper half of transwell (8 μM pore; Costar) membranes were coated with fibronectin (10 μg/ml; Becton Dickinson) overnight at 4° C. to facilitate cell adhesion. Coated membranes were rinsed with PBS and allowed to air dry immediately before use. Cells were detached by trypsinization, and resuspended at a final concentration of 0.5×106 cells/ml in serum-free DMEM containing 0.1% BSA. Cells were added to the upper chamber of the transwell and allowed to migrate toward the bottom chamber containing DMEM, or DMEM supplemented with the chemoattractant bFGF for 4 h in a humidified incubator containing 5% CO₂. Transwell filters were rinsed once with PBS and fixed and stained using a Diff-Quik kit (Baxter) following the manufacturer's protocol. Stained filters were cut out of the chamber and mounted onto slides using Permount (Sigma). The number of migrated cells were measured using microscopy (three fields from each membrane were captured using a 10× objective), and images were captured with a CCD camera using SPOT software. Total migration per membrane was quantified from the captured images using Scion Image software (National Institutes of Health). All experiments were run in triplicate.

Chick Chorioallantoic Membrane (CAM)—The chick CAM assay was conducted as reported previously. Three-day-old chick embryos were removed from their shells and incubated in plastic Petri dishes for 3 days. On embryonic day 6, samples and controls were mixed into methylcellulose and allowed to dry. The discs were applied to the surfaces of developing CAMs, above the dense subectodermal plexus. After an incubation of 48 h, the eggs were examined for vascular reactions under a dissecting scope and photographed. All samples were tested in triplicate for each treatment.

### Results

### Modulators of Angiogenesis in Elasmobranch Cartilage

Using antibody arrays, we have uncovered a panel of angiogenic modulators within elasmobranch cartilage, several promoters of angiogenesis have been confirmed; basic fibroblast growth factor (bFGF), epithelial neutrophil-activating protein 78 (ENA-78), epideimal growth factor (EGF), interleukin 6 (IL-6), monocyte chemoattractant protein-1 (MCP-1), platelet-derived growth factor (PDGF-BB), placental growth factor (PLGF), regulated upon Activation Normal T (RANTES), transforming growth factor (TGF-β1), vascular endothelial growth factor (VEGF), and VEGF-D were found in CCE.

There was positive identification of the angiogenesis stimulators acidic fibroblast growth factor (aFGF), bFGF, granulocyte-colony stimulating factor (G-CSF), hepatocyte growth factor (HGF), IL-6, interleukin 8 (IL-8), Leptin, transforming growth factor (TGFα), and VEGF were made using an antibody array system. Also present in CCE were the angiogenesis inhibitors interleukin 12 (IL-12), interferon-inducible protein 10 (IP-10), and tissue inhibitor of metalloproteinases 1 (TIMP-1).

Using immunoblotting techniques, TnI was found to be present in CCE using anti-rabbit skeletal Tni (Advanced Immunochemicals, Inc.), but the cartilage-derived angiogenesis inhibitors TSP-1, -2 or -3 were not (data not shown). A summary of the results can be seen in Table 1.

TABLE 1
List of angiogenic promoters and inhibitors found in
concentrated cartilage extract of Squalus ancathius.

| Inhibitors | Stimulators | | | | |
|---|---|---|---|---|---|
| Troponin I | aFGF | G-CSF | Leptin | RANTES | VEGF-D |
| IL-12 | bFGF | HGF | MCP-1 | TGFa | |
| IP-10 | EGF | IL-6 | PDGF-BB | TGF-b1 | |
| TIMP-1 | ENA-78 | IL-8 | PLGF | VEGF | |

Purification and Identification of MATN1 from Cartilage

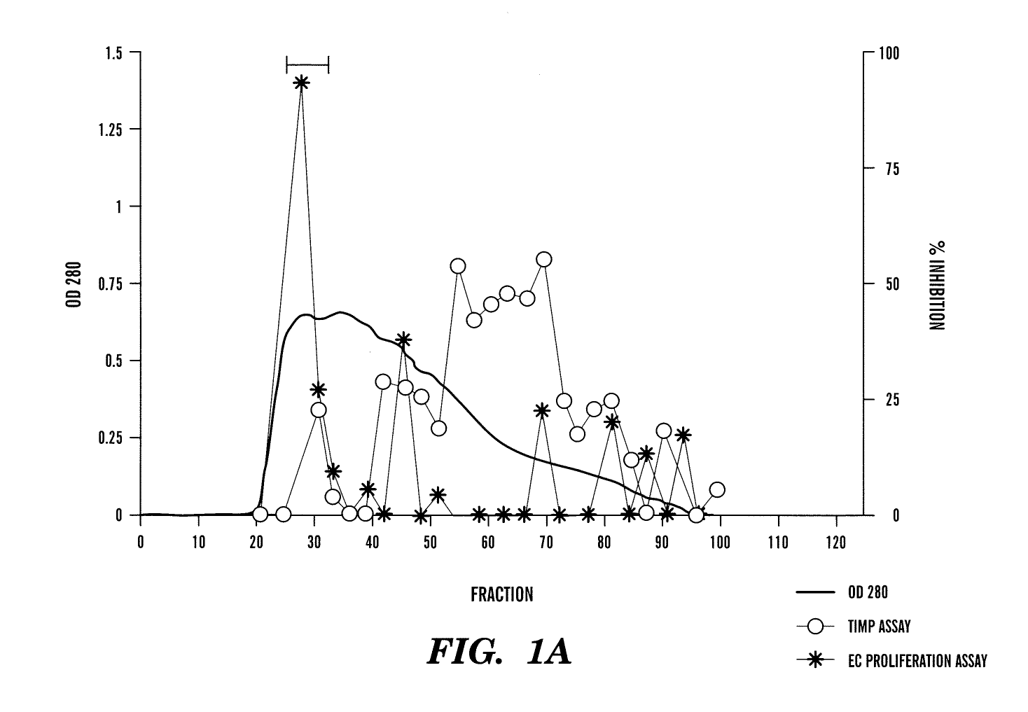

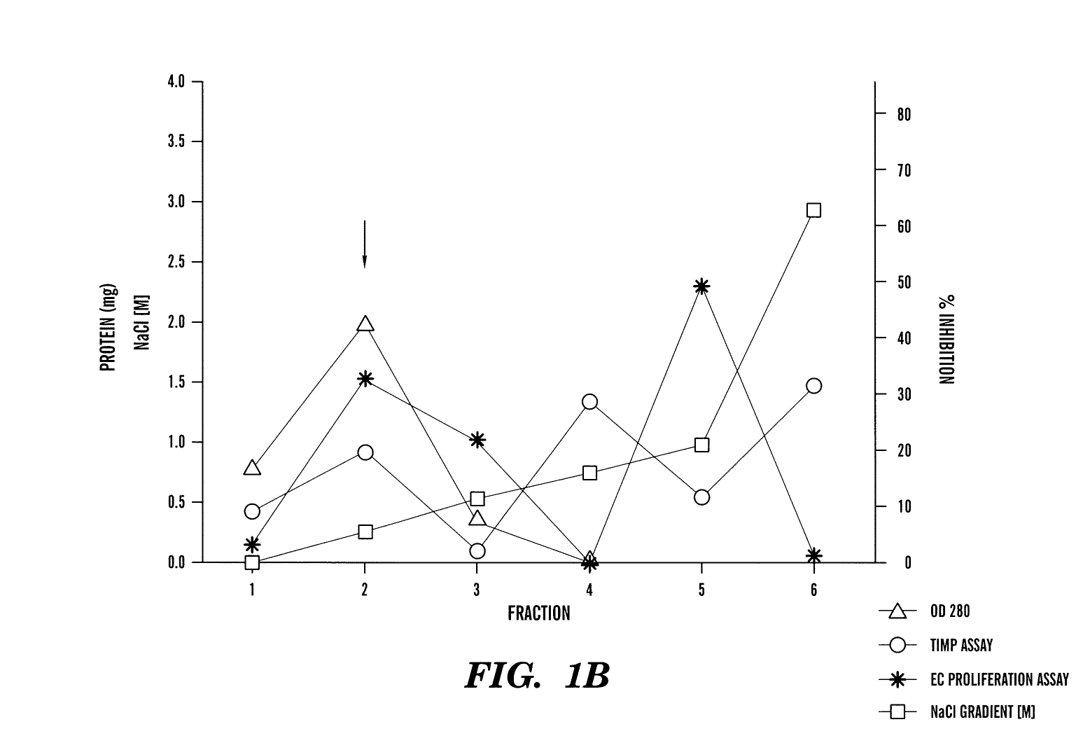

A 3.5 mg sample of CCE, dialyzed against Biogel A-1.5M buffer overnight, was placed in a Biogel A-1.5M size exclusion column (5×50) at a flow rate of 1.0 ml/min, and fractions were collected every 5 minutes (FIGS. 1A and 1B). Every third fraction was screened for the ability to inhibit both EC proliferation and collagenase activity. Fractions 22-34 from the column contained approximately 90% inhibition of capillary EC and a low level of TIMP activity. The fractions were pooled, dialyzed overnight against Bio-Rex buffer to remove the guanidine HCl, then placed in the weak cation exchange Bio-Rex 70 column (Bio-Rad). A scan of all fractions revealed inhibition of EC proliferation in fractions 2 and 5. These specific fractions were dialyzed against Bio-Rex buffer to remove excess salts, then subject to SDS-PAGE in order to see the protein profiles of these fractions, gels were stained with SPYRO Ruby and bands were excised from the gel and analyzed by Mass Spectrometry.

MS/MS revealed a 52 kDa protein-isolated in fraction 2; MALDI-TOF identified two peptides maps indicated two fragments. Monoclonal antibody from Chen's lab identified both the 52 kDa form and the 150 KDa trimer as MATNI.

### Inhibition of Angiogenesis In Vitro by MATN1

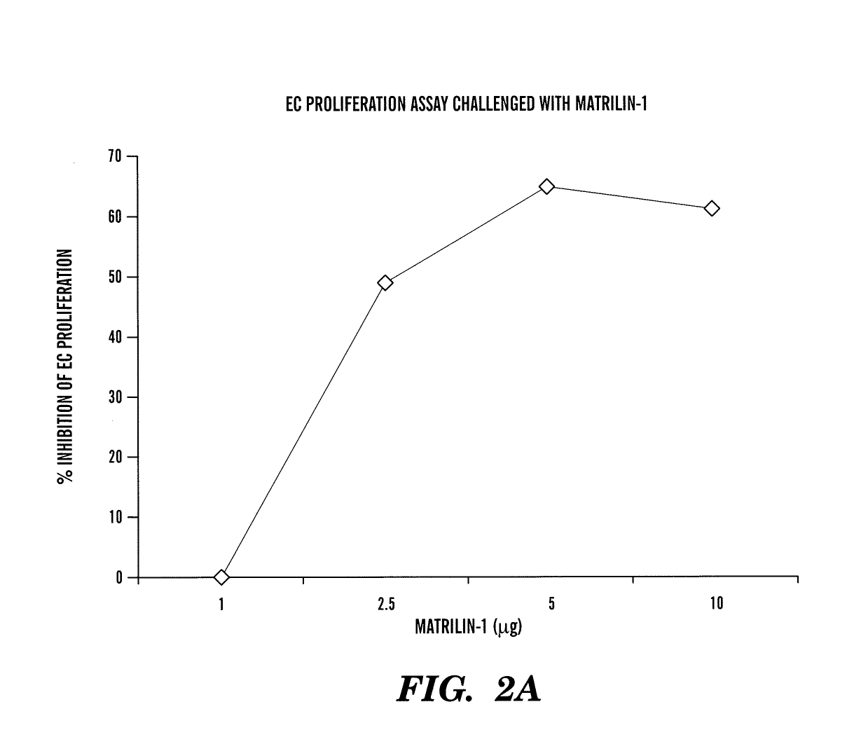

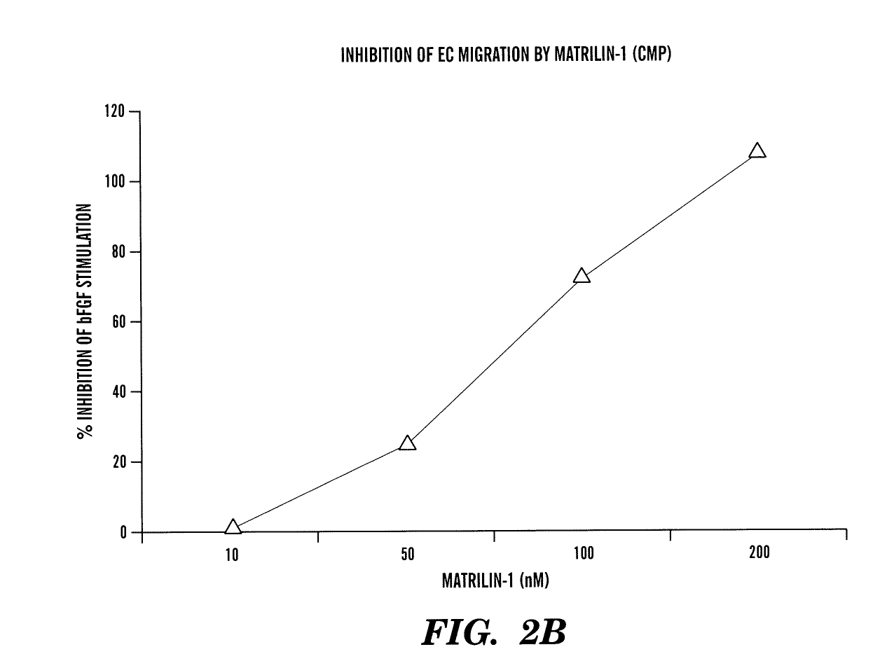

Two assays were used to determine the inhibitory effect of MATN1 against angiogenesis. The first assay tested purified MATN1 for its ability to inhibit bFGF-stimulated capillary EC and was found to inhibit EC proliferation (FIG. 2A). The IC₅₀ of MATN1 was approximately 75 nM. Also, when purified MATN1 was added to the Transwell EC migration assay, migration was inhibited in a dose-dependent manner with an IC₅₀ of approximately 175 mM when bFGF was used as a mitogen (FIG. 2B).

### Inhibition of Angiogenesis In Vivo by MATN1

To determine whether MATN1 was an inhibitor of angiogenesis in vivo, the CAM assay was used. There was significant inhibition of embryonic neovascularization as evidenced by the avascular zone caused by a 5 μg (350 nM) dose of purified MATN1 (data not shown). In contrast, the CAMs containing the 10 μg (700 nM) dose of MATN1 or the control treatment of PBS did not develop avascular zones (data not shown).

In this study, we have examined elasmobranch concentrated cartilage extract (CCE) for known angiogenesis modulators. Also, through a series of chromatographic fractionations of CCE we have identified matrilin-1 as an angiogenesis inhibitor. The in vitro analysis relied on two angiogenesis assays, EC proliferation and migration assays. In vivo, the effect of matrilin-1 on angiogenesis inhibition was investigated using the CAM assay.

Although fractions of shark cartilage such as U-995 (Sheu et al., 1998), SCF2 (Liang and Wong, 2000), SCAIF 80 (Shen et al., 2001), have shown promise inhibiting angiogenesis, as has the ‘angiogenic cocktail’, Neovastat, none of the aforementioned have identified the active biomolecules involved. In the first part of our study, we deployed the use of two angiogenesis antibody arrays and immunoblot analysis using human antibodies to begin to identify some of the present in elasmobranch cartilage (Table 1), including IL-12, IP-10, and TIMP-1. Interestingly enough, there were many angiogenic promoters in CCE. By immunoblotting, we discovered the presence of Troponin I, a known angiogenic inhibitor found in mammalian cartilage (Moses et al., 1999). We did not find the presence of other angiogenic inhibitors that have been isolated from cartilage, such as thrombospondin-1, -2, or -3 (data not shown).

Through a series of size exclusion and cation-exchange chromatographic steps, we began to isolate anti-angiogenic protein from CCE. One protein in particular, Matrilin-1 (MATN1), was identified by MS/MS (tandem mass spectrometry) (data not shown). The matrililns are a family of extracellular matrix proteins (ECM). These modular extra-cellular matrix proteins contain EGF-like, van Willebrand Factor A (vWFA)-like, and alpha helices coiled-coil domains. MATN1, formerly known as cartilage matrix protein (CMP), is an abundant component of tracheal, nasal, auricular, epiphyseal and xiphisternal cartilage (Paulsson and Heinegard, 1982), but is not present in articular cartilage. MATN1 is an example of a pluripotent matrix molecule capable of mediating interactions between a variety of matrix components. It is tightly (covalently) linked to aggrecan chondroiton sulfate. As it can also bind to collagen, it is reasonable to assume it could immobilize chondroiton sulfate-containing proteoglycan fragments in the tissue after their release by aggrecanase or matrix metalloproteinases (Neame et al., 1999).

The discovery of a non-collagenous matrix protein as an angiogenesis inhibitor is not unprecedented. Decorin, an anti-adhesive molecule found in articular cartilage has been shown to suppress cell-mediated angiogenesis (Grant et al., 2002). The results presented here indicate that MATN1 is a useful inhibitor in diseases with unregulated angiogenesis.

### Example II

We have cloned and expressed both Human and Chick Matrilin-1. The chicken and human Matrilin-1 cloning and expression strategies are described in Table 3 and Table 4 respectively.

In order to express cMatrilin-1, all steps described in Table 3 were followed sequentially. The cMatrilin-1 gene was a gift from Dr. Qian Chen in a pCRDNA3.1 plasmid. We first cloned the gene by PCR, with specific sequences on the ends specifically encoding for restriction enzyme cleavage sites. Successful PCR product of cMATN-1 with XloI and XbaI cut sites from pcDNA3.1 plasmid containing cMATN-1 was achieved, 1480 base pairs in length. The PCR product was ligated into a TOPO-TA plasmid in order for the gene to be sequenced; being in the TOPO-TA plasmid also made for a more efficient restriction enzyme double digest. After the TOPO-TA plasmid was transformed into INV110, a dam-E. coli, the double digest worked efficiently. A double digest of the pPICZaC expression plasmid, which contains the same restriction enzyme cleavage sites as the cMatrilin-1 gene, was performed simultaneously. After the restriction enzyme double digest, the expression plasmid was treated with antarcitc phosphatase, which effectively prevents the plasmid from self-ligating. The cMatrilin-1 gene was then ligated into the pPICZaC expression plasmid overnight at 16° C. The pPICZaC plasmid containing the cMatrilin-1 gene was then transformed into GC-10 E. coli. Clones that contained the correct sequence of the cMatrilin-1 gene in the AOX1 promoter region were grown up in large scale in order to obtain a high concentration of plasmid to transform into X-33 wild-type Pichia pastoris. Pichia clones that contained the pPICZaC plasmid containing the cMatrilin-1 gene were then optimized for protein expression.

Proteins expressed from Pichia pastoris clone CF-1E(B) (cMATN1) were run on an SDS-PAGE under reducing and non-reducing conditions. Western blot analysis confirmed of expression of cMATN-1 from Pichia pastoris clone CF-1E(B) (data not shown). Unique steps in cloning Human Matrilin-1 (HuMatrilin-1) are found in Table 4. The HuMatrilin-1 gene was cloned from normal human adult lung trachea cDNA. The HuMatrilin-1 gene was cloned by PCR, with specific sequences on the ends specifically encoding for restriction enzyme cleavage sites. Successful PCR product of HuMATN-1 with XhoI and XbaI cut sites from normal human adult lung trachea cDNA was observed, HuMatrilin-1 is 1396 base pairs in length. The PCR product was ligated into a TOPO-TA plasmid in order for the gene to be sequenced. The rest of the procedure follows exactly that of cMatrilin-1 expression in Table 2.

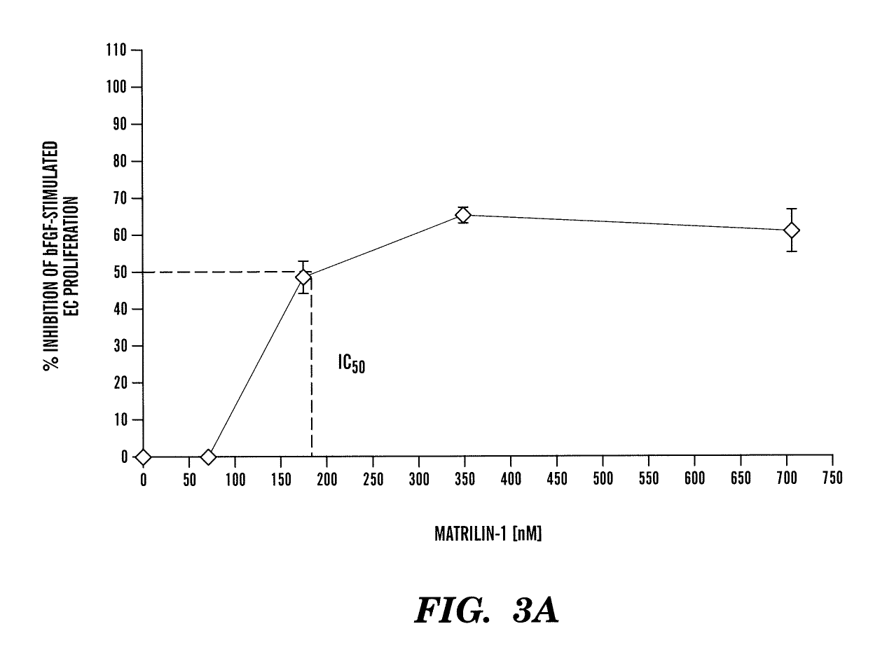

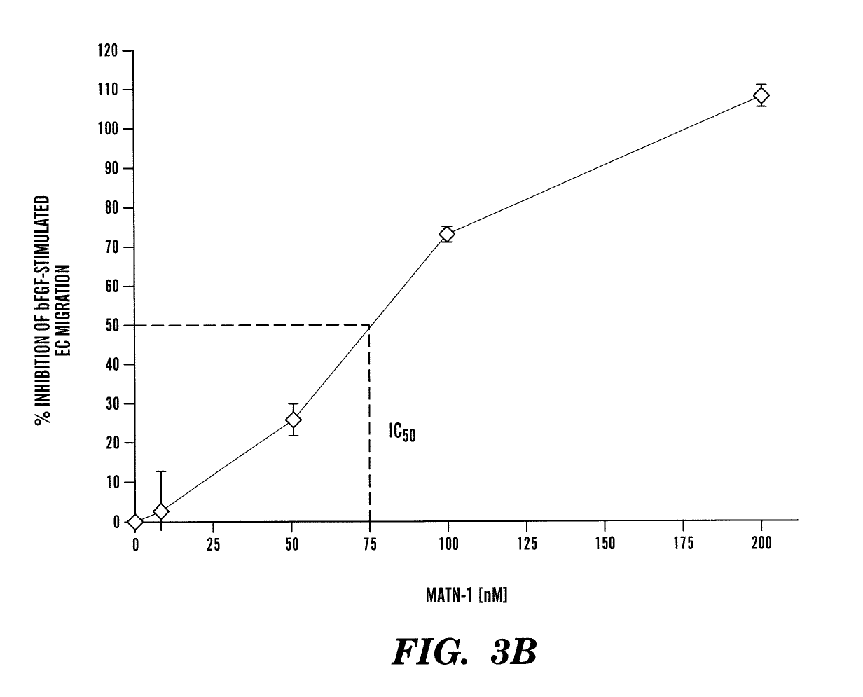

Recombinant chicken and human Matrilin-1 were tested for there ability to inhibit bFGF-stimulated capillary endothelial cells (FIGS. 3A and 3B). Recombinant Chicken Matrilin-1 was found to inhibit EC proliferation in dose-dependent manner, with an IC₅₀ of approximately 275 nM (FIG. 3A). Recombinant Human MATN-1 inhibited EC proliferation with an IC₅₀ Of approximately 190 nM (FIG. 3B).

TABLE 3
Chicken matrilin-1 (cMatrilin-1) cloning and expression strategy

| | |
|---|---|
| Clone chicken Matrilin-1 (cMATN-1) | pPICZαC plasmid |
| from pcDNA3.1 plasmid | |
| TOPO-TA plasmid | ↓ |
| w/cMATN-1 insert | |
| Transform Plasmid into GC-10 E. coli | Transform Plasmid into GC-10 E. coli |
| Miniprep of TOPO-TA plasmid | Miniprep of pPICZαC plasmid |
| w/cMATN-1 insert | |
| Transform plasmids into INV110 E. coli (dam−) | Transform plasmids into INV110 E. coli (dam−) |
| Maxiprep of TOPO-TA plasmid | Maxiprep of pPICZαC plasmid |
| w/cMATN-1 insert (dam−) | |
| gene sequence was confirmed | |

Restriction double digest (XhoI and XbaI) - 1.5 hours @ 37° C.
Gel purification - Freeze 'n Squeeze

| | |
|---|---|
| ↓ | Treat linearized pPICZαC plasmid |
| | With Antarctic Phosphatase |

Ligation of cMATN-1 and pPICZαC via T4 DNA Ligase O/N at 16° C.
Transform plasmid into competent E. coli (GC-10)
Select with Low Salt LB Agarose w/Zeocin plates
Examine 5 colonies from each plate for insertion of gene in correct place
gene sequence was confirmed
Transform pPICZαC expression plasmid containing cMATN-1 into
Pichia pastoris via electroporation
Select for transformed Pichia containing plasmid with YPDS w/Zeocin plates
Examine 5 colonies from each plate for insertion of gene in correct place
Confirmed by MATN-1 primers and alcohol oxidase (AOX1) primers
Optimize cMATN-1 protein expression (FIG. 7, 8)

TABLE 4
Human Matrilin-1 (HuMatrilin-1) cloning and expression strategy

| | |
|---|---|
| Clone human Matrilin-1 (HuMATN-1) from | pPICZαC plasmid |
| normal human trachea tissue | |
| TOPO-TA plasmid | ↓ |
| w/HuMATN-1 insert | |
| Transform Plasmid into GC-10 E. coli | Transform Plasmid into GC-10 E. coli |
| Miniprep of TOPO-TA plasmid | Miniprep of pPICZαC plasmid |
| w/HuMATN-1 insert | |
| Transform plasmids into INV110 E. coli (dam−) | Transform plasmids into INV110 E. coli (dam−) |
| Maxiprep of TOPO-TA plasmid | Maxiprep of pPICZαC plasmid |
| w/HuMATN-1 insert (dam−) | |
| gene sequence was confirmed | |

Restriction double digest (XhoI and XbaI) - 1.5 hours @ 37° C.
Gel purification - Freeze 'n Squeeze

| | |
|---|---|
| ↓ | Treat linearized pPICZαC plasmid |
| | With Antarctic Phosphatase |

Ligation of HuMATN -1 and pPICZαC via T4 DNA Ligase O/N at 16° C.
Transform plasmid into competent E. coli (GC-10)
Plate on Low Salt LB Agarose w/Zeocin
Examine 5 colonies from each plate for insertion of gene in correct place
gene sequence was confirmed
Transform pPICZαC expression plasmid containing cMATN-1 into
Pichia pastoris via electroporation
Select for transformed Pichia containing plasmid with YPDS w/Zeocin plates
Examine 5 colonies from each plate for insertion of gene in correct place
Optimize cMATN-1 protein expression

The references cited herein and throughout the specification are incorporated herein by reference.

### REFERENCES

1. Batist, G., Patenaude, F., Champagne, P., Croteau, D., Levinton, C., Hariton, C., Escudier, B., Dupont, E. 2002. Neovastat (AE-941) in refractory renal cell carcinoma patients: report of a phase II trial with two dose levels. Ann Onco. 13(8):1259-63.
2. Boivin, D., Gendron, S., Beaulieu, E., Gingras, D., Beliveau, R. 2002. The antiangiogenic agent Neovastat (AE-941) induces endothelial cell apoptosis. Mol Cancer Ther. 1(10):795-802.
3. Beliveau, R., Gingras, D., Iruger, E. A., Lamny, S., Sirois, P., Simard, B., Sirois, M. G., Tranqui, L., Baffert, F., Beaulieu, E., Dimitriadou, V., Pepin, M. C., Courjal, F., Ricard, I., Poyet, P., Falardeau, P., Figg W D., Dupont, E. 2002. The antiangiogenic agent neovastat (AE-941) inhibits vascular endothelial growth factor-mediated biological effects. Clin Cancer Res. 8(4):1242-50.
4. Chapman, K. L., Mortier, G. R., Chapman, K., Loughlin, J., Grant, M. E., Briggs, M. D. 2001. Mutations in the region encoding the von Willebrand factor A domain of matrilin-3 are associated with multiple epiphyseal dysplasia. Nat. Genet. 28(4):393-396.
5. Chen, Q., Zhang, Y., Johnson, D. M., Goetinck, P. F. 1999. Assembly of a novel cartilage matrix protein filamentous network: molecular basis of differential requirement of von Willebrand factor A domains. Mol Biol Cell. 10(7): 2149-2162.
6. Deak, F., Piecha, D., Bachrati, C., Paulsson, M., Kiss, I. 1997. Primary structure and expression of matrilin-2, the closest relative of cartilage matrix protein within the von Willebrand factor type A-like module superfamily. J Biol Chem. 272(14): 9268-9274.
7. Deak, F., Wagenerb, R., Kissa, I., Paulssonb, M., 1999. The matrilins: a novel family of oligomneric extracellular matrix proteins. Matrix Biology. 18: 55-64
8. de Lange Davies, C., Melder, R. J., Munn, L. L., Mouta-Carreira, C., Jain, R. K., Boucher, Y.
   2001. Decorin inhibits endothelial migration and tube-like structure formation: role of thrombospondin-1. Microvasc Res. 62(1): 26-42.
9. Gingras, D., Renaud, A., Mousseau, N., Beaulieu, E., Kachra, Z., Beliveau, R. 2001. Matrix proteinase inhibition by AE-941, a multifunctional antiangiogenic compound. Anticancer Res. 21(1A):145-55.
10. Grant, D. S., Yenisey, C., Rose, R. W., Tootell, M., Santra, M., Iozzo, R. V. 2002. Decorin suppresses tumor cell-mediated angiogenesis. Oncogene. 21(31): 4765-4777.
11. Hansson, A. S., Heinegard, D., Holmdahl, R. 1999. A new animal model for relapsing polychondritis, induced by cartilage matrix protein (matrilin-1). J Clin Invest. 104(5): 589-598.
12. Hansson, A. S., Heinegard, D., Piette, J. C., Burkhardt, H., Holmdahl, R. 2001. The occurrence of autoantibodies to matrilin-1 reflects a tissue-specific response to cartilage of the respiratory tract in patients with relapsing polychondritis. Arthritis Rheum. 44(10): 2402-2412.
13. Hiraki, Y., Inoue, H., Iyama, K., Kamizono, A., Ochiai, M., Shukunami, C., Iijima, S., Suzuki, F., Kondo, J. 1997. Identification of chondromliodulin I as a novel endothelial cell growth inhibitor. Purification and its localization in the avascular zone of epiphyseal cartilage. J Biol Chem. 272(51):32419-26.
14. Kusafuka, K., Hiraki, Y., Shukunami, C., Kayano, T., Takemnura, T. 2002. Cartilage-specific matrix protein, chondromodulin-I (ChM-I), is a strong angio-inhibitor in endochondral ossification of human neonatal vertebral tissues in vivo: relationship with angiogenic factors in the cartilage. Acta Histochem. 104(2):167-75.
15. Kang, J. A., Kim, J. T., Song, H. S., Bac, M. K., Yi, E. Y., Kim, K. W., Kim, Y. J. 2003. Anti-angiogenic and anti-tumor invasive activities of tissue inhibitor of metalloproteinase-3 from shark, Scyliorhinus torazame. Biochim Biophys Acta. 1620(1-3):59-64.
16. Kim, J. T., Kim, M. S., Bae, M. K., Song, H. S., Ahn, M. Y., Kim, Y. J., Lee, S. J., Kim, K. W. 2001. Cloning and characterization of tissue inhibitor of metalloproteinase-3 (TIMP-3) fromi slarlk, Scyliorhinus torazam. Bioclhim Biophys Acta. 1517(2):311-5.
17. Klatt, A. R., Nitsclhe, D. P., Kobbe, B., Maclit, M., Paulsson, M., Wagener, R. 2001. Molecular structure, processing, and tissue distribution of matrilin-4. J Biol. Chem. 276(20): 17267-17275.
18. Lee, A., Langer, R. 1983. Shark cartilage contains inhibitors of tumor angiogenesis. Science. 221(4616):1185-1187.
19. Liang, J. H., Wong, K. P. 2000. The characterization of angiogenesis inhibitor from shark cartilage. Adv Exp Med. Biol. 476:209-23.
20. Makihira, S., Yan, W., Olmo, S., Kawamotoi, T., Fujimotoi, K., Okimura, A., Yoshidai, E., Noshiroi, M., Hamada, T., Katoi, Y., 1999. Enhancement of Cell Adhesion and Spreading by a Cartilage-specific Noncollagenous Protein, Cartilage Matrix Protein (CMP/Matrilin-1), via Integrin α1β1. J. Biol. Chem. 274(16): 11417-11423.
21. McAdam, L. P., O'Hanlan, M. A., Bluestone, R., Pearson, C. M. 1976. Relapsing polychondritis:
22. prospective study of 23 patients and a review of the literature. Medicine. 55(3):193-215.
23. Michet, C. J., McKenna, C. H., Luthra, H. S., O'Fallon, W. M. 1986. Relapsing polychondritis. Survival and predictive role of early disease manifestations. Ann Intern Med. 104(1): 74-78.
24. Moses, M. A., Sudhalter, J., Langer, R. 1990. Identification of an inhibitor of neovascularization from cartilage. Science. 248(4961):1408-10.
25. Moses, M. A., Sudhalter, J., Langer, R. 1992. Isolation and characterization of an inhibitor of neovasculalization from scapular chondrocytes. J. Cell Biol. 119(2):475-82.
26. Moses, M. A., Wiederschain, D., Wu, I., Fernandez, C. A., Ghazizadeh, V., Lane, W. S., Flynn, E., Sytkowski, A., Tao, T., Langer, R. 1999. Troponin I is present in human cartilage and inhibits angiogenesis. Proceedings of the National Academy of Sciences. 96: 2645-2650.
27. Mostert, A. K., Dijkstra, P. F., Jansen, B. R., van Horn, J. R., de Graaf, B., Heutink, P., Lindhout, D. 2003. Familial multiple epiphyseal dysplasia due to a matrilin-3 mutation: further delineation of the phenotype including 40 years follow-up. Am J Med. Genet. 120A(4):490-497.
28. Neamie, P. J., Tapp, H., Azizan, A. 1999. Noncollagenous, nonproteoglycan macromolecules of cartilage. Cell Mol Life Sci. 55(10):1327-1340.
29. Oshima, Y., Sato, K., Tashiro, F., Miyazki, J., Nislhida, K., Hiraki, Y., Tano, Y., Shukunamni, C. 2004. Anti-angiogenic action of the C-terminal domain of tenomnodulin that shares homology with chondromodulin-1. J Cell Science. 117 (13): 2731-2744.
30. Paulsson, M., Heinegard, D. 1979. Matrix proteins bound to associatively prepared proteoglycans from bovine cartilage. Biochem J. 183(3): 539-545.
31. Paulsson, M., Heinegard, D. 1982. Radioimmunoassay of the 148-kilodalton cartilage protein. Distribution of the protein among bovine tissues. Biochem J. 207(2): 207-213.
32. Piecha, D., Muratoglu, S., Morgelin, M., Hauser, N., Studer, D., Kiss, I., Paulsson, M., Deak, F. 1999. Matrilin-2, a large, oligomeric matrix protein, is expressed by a great variety of cells and forms fibrillar networks. J Biol. Chem. 274(19): 13353-13361.
33. Saxne, T., Heingard, D. 1995. Serum-concentrations of two cartilage matrix proteins reflecting different aspects of cartilage turnover in relapsing polychondritis. Arthritis Rheum. 38: 294-296.
34. Senger, D. R., Claffey, K. P., Benes, J. E., Perruzzi, C. A., Sergiou, A. P., Detmar, M., 1997. Angiogenesis promoted by vascular endotlhelial growth factor: Regulation through a1b1 integrins. Proc. Nat. Acad. Sci. USA. 94: 13612-13617.
35. Shen, X. R., Ji, D. M., Hu, Y. Q., Jia, F. X., Wang, L., Chu, Z. Y., Ren, D. M. 2001. SCAIF80, a Novel Inhibitor of Angiogenesis, and Its Effect on Tumor Growth. Sheng Wu Hua Xue Yu Sheng Wu Wu Li Xue Bao (Shanghai). 33(1):99-104.
36. Sheu, J. R., Fu, C. C., Tsai, M. L., Chung, W. J. 1998. Effect of U-995, a potent shark cartilage-derived angiogenesis inhibitor, on anti-angiogenesis and anti-tumor activities. Anticancer Res. 18(6A):4435-41.
37. Sumiipio, B. E., Riley, J. T., Dardik, A., 2002. Molecules in focus Cells in focus: endothelial cell. The International Journal of Biochemistry & Cell Biology. 34: 1508-1512.
38. Tsonis, P. A., Goetinck, P. F. 1988. Expression of cartilage-matrix genes and localization of their translation products in the embryonic click eye. Exp Eye Res. 46(5):753-764.
39. Wiberg, C., Klatt, A. R., Wagener, R., Paulsson, M., Baternan, J. F., Heinegard, D., Mörgelin, M., 2003. Comiplexes of Matrilin-1 and Biglycan or Decorin Connect Collagen VI Microfibnils to Both Collagen II and Aggrecan. J. Biol. Chem. 278(39): 37698-37704,
40. Zhang, Y., Chen, Q., 2000. Changes of Matrilin Forms during Endochondral Ossification. MOLECULAR BASIS OF OLIGOMERIC ASSEMBLY. J. Biol. Chem. 275(42):32628-32634.

SEQUENCES
Homo Sapiens Matrilin-1 Protein
(SEQ ID NO: 1)
MRVLSGTSLMLCSLLLLLQALCSPGLAPQSRGHLCRTRPTDLVF
VVDSSRSVRPVEFEKVKVFLSQVIESLDVGPNATRVGMVNYASTVKQEFSLRAHVSKA
ALLQAVRRIQPLSTGTMTGLAIQFAITKAFGDAEGGRSRSPDISKVVIVVTDGRPQDS
VQDVSARARASGVELFAIGVGSVDKATLRQIASEPQDEHVDYVESYSVIEKLSRKFQE
AFCVVSDLCATGDHDCEQVCISSPGSYTCACHEGFTLNSDGKTCNVCSGGGGSSATDL
VFLIDGSKSVRPENFELVKKFISQIVDTLDVSDKLAQVGLVQYSSSVRQEFPLGRFHT
KKDIKAAVRNMSYMEKGTMTGAALKYLIDNSFTVSSGARPGAQKVGIVFTDGRSQDYI
NDAAKKAKDLGFKMFAVGVGNAVEDELREIASEPVAEHYFYTADFKTINQIGKKLQKK
ICVEEDPCACESLVKFQAKVEGLLQALTRKLEAVSKRLAILENTVV
Chick Matrilin-1 Protein
(SEQ ID NO: 2)
MDGIFCALPL SLLLLLQSCG VWGAPPQPRG TLCRTKPTDL VFIIDSSRSV
RPQEFEKVKV FLSRVIEGLD VGPNSTRVGV INYASAVKNE FSLKTHQTKA
ELLQAVQRIE PLSTGTMTGL AIQFAISRAF SDTEGARLRS PNINKVAIVV
TDGRPQDGVQ DVSARARQAG IEIFAIGVGR VDMHTLRQIA SEPLDDHVDY
VESYSVIEKL THKFQEAFCV VSDLCATGDH DCEQICISTP GSYKCACKEG
FTLNNDGKTC SACSGGSGSA LDLVFLIDGS KSVRPENFEL VKKFINQIVE
SLEVSEKQAQ VGLVQYSSSV RQEFPLGQFK NKKDIKAAVK KMAYMEKGTM
TGQALKYLVD SSFSIANGAR PGVPKVGIVF TDGRSQDYIT DAAKKAKDLG
FRMFAVGVGN AVEDELREIA SEPVAEHYFY TADFRTISNI GKKLQMKICV
EEDPCECKSI VKFQTKVEEL INTLQQKLEA VAKRIEALEN KII
Homo sapiens (human)MATRILIN-2
(SEQ ID NO: 3)
MEKMLAGCFLLILGQIVLLPAEARERSRGRSISRGRHARTHPQTALLESSCENKRADLVF
IIDSSRSVNTHDYAKVKEFIVDILQFLDIGPDVTRVGLLQYGSTVKNEFSLKTFKRKSEVE
RAVKRMRHLSTGTMTGLAIQYALNIAFSEAEGARPLRENVPRVIMIVTDGRPQDSVAEV
AAKARDTGILIFAIGVGQVDFNTLKSIGSEPHEDHVFLVANFSQIETLTSVFQKKLCTAH
MCSTLEHNCAHFCINIPGSYVCRCKQGYILNSDQTTCRIQDLCAMEDHNCEQLCVNVPG
SFVCQCYSGYALAEDGKRCVAVDYCASENHGCEHECVNADGSYLCQCHEGFALNPDK
KTCTKIDYCASSNHGCQHECVNTDDSYSCHCLKGFTLNPDKKTCRRINYCALNKPGCEH
ECVNMEESYYCRCHRGYTLDPNGKTCSRVDHCAQQDHGCEQLCLNTEDSFVCQCSEGF
LINEDLKTCSRVDYCLLSDHGCEYSCVNMDRSFACQCPEGHVLRSDGKTCAKLDSCAL
GDHGCEHSCVSSEDSFVCQCFEGYILREDGKTCRRKDVCQAIDHGCEHICVNSDDSYTC
ECLEGFRLAEDGKRCRRKDVCKSTHHGCEHICVNNGNSYICKCSEGFVLAEDGRRCKK
CTEGPIDLVFVIDGSKSLGEENFEVVKQFVTGIIDSLTISPKAARVGLLQYSTQVHTEFTLR
NFNSAKDMKKAVAHMKYMGKGSMTGLALKHMFERSFTQGEGARPLSTRVPRAAIVFT
DGRAQDDVSEWASKAKANGITMYAVGVGKAIEEELQEIASEPTNKHLFYAEDFSTMDEI
SEKLKKGICEALEDSDGRQDSPAGELPKTVQQPTESEPVTINIQDLLSCSNFAVQHRYLFE
EDNLLRSTQKLSHSTKPSGSPLEEKHDQCKCENLIMFQNLANEEVRKLTQRLEEMTQRM
EALENRLRYR
Homo sapiens (human)MATRILIN-3
(SEQ ID NO: 4)
MPRPAPARRLPGLLLLLWPLLLLPSAAPDPVARPGFRRLETRGPGGSPGRRPSPAAPDGA
PASGTSEPGRARGAGVCKSRPLDLVFIIDSSRSVRPLEFTKVKTFVSRIIDTLDIGPADTRV
AVVNYASTVKIEFQLQAYTDKQSLKQAVGRITPLSTGTMSGLAIQTAMDEAFTVEAGAR
EPSSNIPKVAIIVTDGRPQDQVNEVAARAQASGIELYAVGVDRADMASLKMMASEPLEE
HVFYVETYGVIEKLSSRFQETFCALDPCVLGTHQCQHVCISDGEGKHHCECSQGYTLNA
DKKTCSALDRCALNTHGCEHICVNDRSGSYHCECYEGYTLNEDRKTCSAQDKCALGTH
GCQHICVNDRTGSHHCECYEGYTLNADKKTCSVRDKCALGSHGCQHICVSDGAASYHC
DCYPGYTLNEDKKTCSATEEARRLVSTEDACGCEATLAFQDKVSSYLQRLNTKLDDILE
KLKINEYGQIHR
Homo sapiens (human)MATRILIN-4
(SEQ ID NO: 5)
MRGLLCWPVLLLLLQPWETQLQLTGPRCHTGPLDLVFVIDSSRSVRFEFETMRQFLMG
LLRGLNVGPNATRVGVIQYSSQVQSVFPLRAFSRREDMERAIRDLVPLAQGTMTGLAIQ
YAMNVAFSVAEGARPPEERVPRVAVIVTDGRPQDRVAEVAAQARASGIEIYAVGVQRA
DVGSLRAMASPPLDEHVFLVESFDLIQEFGLQFQSRLCAIDLCAEGTHGCEHHCVNSPGS
YFCHCQVGFVLQQDQRSCRAIDYCSFGNHSCQHECVSTPGGPRCHCREGHDLQPDGRS
CQVRDLCNGVDHGCEFQCVSEGLSYRCLCPEGRQLQADGKSCNRCREGHVDLVLLVD
GSKSVRPQNFELVKRFVNQIVDFLDVSPEGTRVGLVQFSSRVRTEFPLGRYGTAAEVKQ
AVLAVEYMERGTMTGLALRHMVEHSFSEAQGARPRALNVPRVGLVFTDGRSQDDISV
WAARAKEEGIVMYAVGVGKAVEAELREIASEPAELHVSYAPDFGTMTHLLENLRGSICP
EEGISAGTELRSPCECESLVEFQGRTLGALESLTLNLAQLTARLEDLENQLANQK

1. A method for treatment of a disease that responds to all inhibition of angiogenesis comprising administering to a mammal a pharmaceutical composition comprising an effective angiogenesis-inhibiting amount of a Matrilin-1, wherein the composition is substantially free of other cartilage proteins.

2. The method of claim 1, wherein the disease that responds to an inhibition of angiogenesis is cancer.

3. The method of claim 1, wherein Matrilin-1 is human Matrilin-1 and comprises amino acids 1-496 of SEQ ID NO.: 1.

4. The method of claim 3, wherein Matrilin-1 comprises amino acids 23-222 of SEQ ID NO.: 1.

5. The method of claim 3, wherein Matrilin-1 comprises amino acids 43-222 of SEQ ID NO.: 1.

6. The method of claim 3, wherein Matrilin-1 comprises amino acids 264-453 of SEQ ID NO.: 1.

7. The method of claim 3, wherein Matrilin-1 comprises amino acids 278-453 SEQ ID NO.: 1.

8. The method of claim 1, wherein Matrilin-1 is Shark Matrilin-1.

9. The method of claim 1, wherein Matrilin-1 is Chick Matrilin-1 and comprises amino acids 1-493 of SEQ ID NO.: 2.

10. The method of claim 9, wherein Matrilin-1 comprises amino acids 24-220 of SEQ ID NO.: 2.

11. The method of claim 9, wherein Matrilin-1 comprises amino acids 42-220 of SEQ ID NO.: 2.

12. The method of claim 9, wherein Matrilin-1 comprises amino acids 262-450 of SEQ ID NO.: 2.

13. The method of claim 9, wherein Matrilin-1 comprises amino acids 275-450 of SEQ ID NO.: 2.

14. The method of claim 1, wherein Matrilin-1 comprises a Matrilin-1 derivative having at least 50% identity compared to a fragment of Matrilin-1 from which the derivative was derived, wherein the derivative is derived from SEQ ID NO: 1, or SEQ ID NO: 2.

15. The method of claim 1, wherein Matrilin-1 is recombinant Matrilin-1.

16. The method of claim 1, wherein said disease that responds to an inhibition of angiogenesis is retinopathy of prematurity, diabetic retinopathy or macular degeneration.

17. The method of claim 1, wherein said disease the responds to an inhibition of angiogenesis is arthritis or rheumatoid arthritis.

18. The method of claim 1, wherein said administering is conducted in conjunction with chemotherapy.

19. The method of claim 1, wherein said administering is conducted in conjunction with radiation therapy.

20. The method of claim 1, wherein said administering is conducted in conjunction with a second angiogenesis inhibitor.

21. The method of claim 1, wherein said mammal is at risk for developing said disease the responds to an inhibition of angiogenesis.

22. The method of claim 1, wherein said Matrilin-1 is incorporated into a stent for local release and inhibition of restenosis.

23. A medical device comprising Matrilin-1.

24. The medical device of claim 1, wherein the device is a stent, catheter, cannula, or an electrode.

25. A pharmaceutical composition comprising Matrilin-1, wherein the composition is substantially free of other cartilage proteins.

26. The composition of claim 25, wherein the composition is formulated for sustained release.

27. The composition of claim 25, wherein the composition is formulated for delivery by an osmotic pump.
